SEQUENCE LISTING (2 sequences)

SEQ ID NO: 1 - protein, 132 aa, Homo sapiens
MTGELEVKNMDMKPGSTLKITGSIADGTDGFVINLGQGTDKLNLHFNPRFSESTIVCNSLDGSNWGQEQREDHLCFSPGSEVKFTVTFESDKFKVKLPDGHELTFPNRLGHSHLSYLSVRGGFNMSSFKLKE

SEQ ID NO: 2 - protein, 130 aa, Rattus norvegicus
MSEKFEVTNLNMKSGMSLKIKGKIHNDVNSFTINLGQGKETLNLHFNPRFNESTIVCNTLDGSSWGQEQRENHSCFSPGSEVKLTLTFQDKDFKVTLPDGHSLTFPNRLGHNHLRYLSMDGLQISSFKLE

(19) United States
(12) Patent Application Publication
(10) Pub. No.: US 2008/0113907 A1
(43) Pub. Date: May 15, 2008

(54) Uses Of Galectin-2

(75) Inventors: Stefan Rosewicz, Berlin (DE); Ehrengard Rosewicz, Berlin (DE); Axel Dignass, Berlin (DE); Andreas Sturm, Berlin (DE)

Correspondence Address: Davidson, Davidson & Kappel, LLC, 485 7th Avenue, 14th Floor, New York, NY, 10018, US

(73) Assignee: Charite-Universitat-Smedizin Berlin, Berlin (DE)

(21) Appl. No.: 10/593,533

(22) PCT Filed: Mar. 24, 2005
(86) PCT No.: PCT/EP05/03161

§ 371 Date: Jul. 11, 2007

(30) Foreign Application Priority Data
Mar. 26, 2004 (EP) 04007345.4
Aug. 19, 2004 (EP) 04 019 696.6

(51) Int. Cl. A61K 38/17; A61K 48/00

(52) U.S. Cl. 514/12; 514/44; 514/8

(57) ABSTRACT

Use of galectin-2 or of a nucleic acid coding for galectin-2 or of its complementary strand, or of a nucleic acid hybridizing to such coding nucleic acid or its complementary strand, for the manufacture of a medicament for the treatment or prevention of a patient having a disease with impaired apoptosis of T-cells, macrophages and/or antigen-presenting cells, or for the manufacture of a medicament for the treatment or prevention of organ rejection in a patient having undergone organ transplantation, in particular solid organ transplantation.

The present invention relates to uses of galectin-2.

The central function of the immune system of higher animals is the distinction between foreign entities, such as infectious agents, bacteria, viruses etc., from self components of the body. The immune systems is a dynamic system showing a plastic behaviour which means that the immune system is a learning system throughout the entire life span of an individual. During the development of the body, usually, the immune system acquires the ability to tolerate self components whilst developing and maintaining the capability of recognizing foreign components. The acquisition of self tolerance occurs by clonal deletion of autoreactive T-cells by apoptosis in the thymus gland during the perinatal period, and by functionally suppressing autoreactive T and B cells at later stages of the development. At present, it is not entirely clear how the system of self tolerance is maintained or how, under specific circumstances, it breaks down. Sometimes the organism loses its capability of self tolerance and the organism's immune system fails to discriminate between self and non-self antigens, as a result of which an autoimmune response is launched which manifests itself in the activation and clonal expansion of autoreactive lymphocytes and the production of auto-antibodies against autologous antigens of normal body tissues. There exist different animal models for various autoimmune diseases, but from these it is not clear what causes the onset of an autoimmune response nor, in many cases, what the further course of events after an initial autoimmune response will be. Many of the autoimmune diseases are rare. Multifactorial in origin, autoimmune diseases occur more frequently in individuals with a genetic pre-disposition. They may be caused by specific weaknesses of immune regulatory controls, certain environmental factors, etc. the combination of which may ultimately lead to the immune system launching an attack against self components.

Autoimmune diseases can be classified into preferably systemic and preferably organ-specific diseases. In systemic autoimmune diseases tissue injury and inflammation occurs in multiple sites in organs without relation to their antigenic make up. In many instances such systemic plexes which are formed by auto-antibody responses to ubiquitous soluble cellular antigens. Typical examples of these systemic autoimmune diseases are systemic lupus erythematodes, scleroderma, polymyositis, rheumatoid arthritis and ankylosing spondylitis. Organ-specific autoimmune diseases, on the other hand, can be further classified according to the organ they affect. Autoimmune diseases of the nervous system include multiple sclerosis, myasthenia gravis, autoimmune neuropathies, such as Guillain-Barré. Autoimmune diseases affecting the gastrointestinal system are Crohn's disease, ulcerative colitis, primary biliary cirrhosis, primary sclerosing cholangitis, sprue's disease, auto-immune enteropathy, and autoimmune hepatitis. Autoimmune diseases effecting endocrine glands are diabetes mellitus type 1, Grave's disease, Hashimoto's thyroiditis and autoimmune oophoritis.

Crohn's disease (CD) is an autoimmune disease which c an affect any part of the digestive tract, from the mouth to the anus. However, it usually occurs in the lower part of the small intestine. It manifest itself in a severe inflammation extending deep into the lining of the intestine. The symptoms of Crohn's disease are abdominal pain, diarrhea, rectal bleeding, weight loss and fever. The etiology of Crohn's disease remains unclear. Diagnosis usually is made by the presentation of typical clinical symptoms, and a combination of blood tests, colonoscopy including biopsies, x-ray examinations with contrasting agents, magnetic resonance imaging or CT. Treatment of Crohn's disease includes anti-inflammatory agents such as mesalazine, sulfasalazine, corticosteroids and immunosuppressants. The latter include 6-mercaptopurine and azathioprine. Recently a monoclonal antibody (mAb) infliximab has been authorized for the treatment of moderate to severe or fistulizing Crohn's disease which does not respond to standard therapies, such as mesalasazine, corticosteroids and/or immunosuppressive agents and antibiotics in fistulizing CD. Infliximab which is the first agent approved specifically for RA and Crohn's disease is a chimeric antibody against tumor necrosis factor α (anti-TNF α). TNF α is a cytokine produced by the immune system and plays a crucial role in the pathogenesis of Crohn's disease. Treatment of patients with CD induces apoptosis of lamina propria T-cells and macrophages located in the intestinal mucosa or joints. However, during treatment with infliximab, frequently other antibodies are generated which attenuate the action of the anti-TNF α-antibody (New England Journal of Medicine 2003; 348:601-608) and cause intolerances (type I hypersensitivity). Furthermore, infliximab appears to suppress the immune system in general, thus exposing the organism to secondary super infections and therefore causing sepsis, tuberculosis and death.

Ulcerative colitis is a disease that causes inflammation and sores, called ulcers, in the lining of the large intestine. The inflammation usually occurs in the rectum and lower part of the colon, but it may affect the entire colon. Ulcerative colitis rarely affects the small intestine except for the end section, called the terminal ileum. Ulcerative colitis may also be called colitis or proctitis.

The inflammation makes the colon empty frequently, causing diarrhea. Ulcers form in places where the inflammation has killed the cells lining the colon; the ulcers bleed and produce pus.

Ulcerative colitis is an inflammatory bowel disease (IBD), the general name for diseases that cause inflammation in the small intestine and colon. Ulcerative colitis can be difficult to diagnose because its symptoms are similar to other intestinal disorders as i.e. Crohn's disease. Ulcerative colitis may occur in people of any age, but most often it starts between ages 15 and 30, or less frequently between ages 50 and 70. Children and adolescents sometimes develop the disease. Ulcerative colitis affects men and women equally and appears to run in some families.

Theories about what causes ulcerative colitis abound, but none have been proven. The most popular theory is that the body's immune system reacts to a virus or a bacterium by causing ongoing inflammation in the intestinal wall.

People with ulcerative colitis have abnormalities of the immune system, but doctors do not know whether these abnormalities are a cause or a result of the disease. Ulcerative colitis is not caused by emotional distress or sensitivity to certain foods or food products, but these factors may trigger symptoms in some people.

The most common symptoms of ulcerative colitis are abdominal pain and bloody diarrhea. Patients also may experience fatigue, weight loss, loss of appetite, rectal bleeding, loss of body fluids and nutrients

About half of patients have mild symptoms. Others suffer frequent fever, bloody diarrhea, nausea, and severe abdominal cramps. Ulcerative colitis may also cause problems such as arthritis, inflammation of the eye, liver disease (hepatitis, cirrhosis, and primary sclerosing cholangitis), osteoporosis, skin rashes, and anemia. No one knows for sure why problems occur outside the colon. Scientists think these complications may occur when the immune system triggers inflammation in other parts of the body. Some of these problems go away when the colitis is treated.

A thorough physical exam and a series of tests may be required to diagnose ulcerative colitis.

Blood tests may be done to check for anemia, which could indicate bleeding in the colon or rectum. Blood tests may also uncover a high white blood cell count, which is a sign of inflammation somewhere in the body. By testing a stool sample, the doctor can detect bleeding or infection in the colon or rectum.

The doctor may do a colonoscopy or sigmoidoscopy to be able to see any inflammation, bleeding, or ulcers on the colon wall. During the exam, the doctor may do a biopsy, which involves taking a sample of tissue from the lining of the colon to view with a microscope. A barium enema x ray of the colon may also be required. This procedure involves filling the colon with barium, a chalky white solution. The barium shows up white on x ray film, allowing the doctor a clear view of the colon, including any ulcers or other abnormalities that might be there.

Treatment for ulcerative colitis depends on the seriousness of the disease. Most people are treated with medication. In severe cases, a patient may need surgery to remove the diseased colon. Surgery is until now the only cure for ulcerative colitis.

Some people whose symptoms are triggered by certain foods are able to control the symptoms by avoiding foods that upset their intestines, like highly seasoned foods, raw fruits and vegetables, or milk sugar (lactose). Each person may experience ulcerative colitis differently, so treatment is adjusted for each individual. Emotional and psychological support is important.

Some people have remissions—periods when the symptoms go away—that last for months or even years. However, most patients' symptoms eventually return. This changing pattern of the disease means one cannot always tell when a treatment has helped.

Some people with ulcerative colitis may need medical care for some time, with regular doctor visits to monitor the condition.

The goal of therapy is to induce and maintain remission, and to improve the quality of life for people with ulcerative colitis. Several types of drugs are available.

Aminosalicylates, drugs that contain 5-aminosalicyclic acid (5-ASA), help control inflammation. Sulfasalazine is a combination of sulfapyridine and 5-ASA and is used to induce and maintain remission. The sulfapyridine component carries the anti-inflammatory 5-ASA to the intestine. However, sulfapyridine may lead to side effects such as include nausea, vomiting, heartburn, diarrhea, and headache. Other 5-ASA agents such as olsalazine, mesalamine, and balsalazide, have a different carrier, offer fewer side effects, and may be used by people who cannot take sulfasalazine. 5-ASAs are given orally, through an enema, or in a suppository, depending on the location of the inflammation in the colon. Most people with mild or moderate ulcerative colitis are treated with this group of drugs first.

Corticosteroids such as prednisone and hydrocortisone also reduce inflammation. They may be used by people who have moderate to severe ulcerative colitis or who do not respond to 5-ASA drugs. Corticosteroids, also known as steroids, can be given orally, intravenously, through an enema, or in a suppository, depending on the location of the inflammation. These drugs can cause side effects such as weight gain, acne, facial hair, hypertension, mood swings, and an increased risk of infection. For this reason, they are not recommended for long-term use.

Immunomodulators such as azathioprine and 6-mercapto-purine (6-MP) reduce inflammation by affecting the immune system. They are used for patients who have not responded to 5-ASAs or corticosteroids or who are dependent on corticosteroids. However, immunomodulators are slow-acting and may take up to 6 months before the full benefit is seen. Patients taking these drugs are monitored for complications including pancreatitis and hepatitis, a reduced white blood cell count, and an increased risk of infection. Cyclosporine A may be used with 6-MP or azathioprine to treat active, severe ulcerative colitis in people who do not respond to intravenous corticosteroids.

Other drugs may be given to relax the patient or to relieve pain, diarrhea, or infection.

Rheumatoid arthritis (RA) is also believed to be an autoimmune disease. Rheumatoid arthritis causes inflammation and deformity of the joints. Other problems throughout the body may also develop, including inflammation of blood vessels, the development of rheumatoid arthritic nodules and osteoporosis. Rheumatoid arthritis is considered an autoimmune disease which is acquired and in which genetic factors appear to play a role. The presence of HLA-DR4 antibody in 70 percent of patients with RA lends support to the genetic predisposition to the disease. Patients having rheumatoid arthritis, in many instances also show rheumatoid factors (RF), which are antibodies to IgG and which are present in 60-80 percent of adults with the disease. High titers of rheumatoid factors are usually associated with more severe and active joint disease, greater systemic involvement and a poorer prognosis for remission. Rheumatoid arthritis, as well as other autoimmune diseases, includes widespread immunologic and inflammatory alterations of connective tissue. Because the autoimmune diseases share many clinical findings, making a differential diagnosis is often difficult. The prevalence of the disease is 1-2 percent of the general population and is found world-wide. There seems to be a certain pre-disposition in women which show a 3:1 ratio to men with respect to the occurrence and onset of the disease. The disease usually manifests itself in adults between the age of 40 to 60 years. The etiology of rheumatoid arthritis remains unknown. Metabolic and nutritional factors, the endocrine system, geographic, psychologic and occupational data have been extensively studied with no conclusive findings. There is a number of etiological theories on rheumatoid arthritis, some of which postulate an unknown antigen which initiates the autoimmune response. Others suspect an infectious origin of the disease process, which has included various bacteria and viruses but without evidence of precipitating events. Again, rheumatoid arthritis is diagnosed using standard criteria (ARA) (American Rheumatism Association—see also for example http://www.shim.org/rheumatology/1987ra.html) including a number of tests, including blood tests, in particular tests for the erythrocyte sedimentation rate, tests for anemia and tests for Rheumatoid factor. Furthermore, because rheumatoid arthritis in particular affects synovial joints, the synovial fluid is examined. When rheumatoid arthritis is present, the fluid has an increased protein content and a decreased or normal glucose content. Furthermore it contains a higher than normal number of white blood cells. Usually diagnosis of rheumatoid arthritis is a combination of positive results of various of the aforementioned tests. Rheumatoid arthritis can be treated in a number of ways, although there is no cure up to date. Rheumatoid arthritis may be treated by non-steroidal anti-inflammatory agents, by anti-rheumatic drugs, including penicillamine and cyclo-oxygenase-2-inhibitors. Furthermore, like Crohn's disease, rheumatoid arthritis is also being treated by antibodies directed against TNF α, such as infliximab, ethanercept and adalimumab. Additionally, quite recently an IL-1 receptor antagonist, anakinra, has been market authorized for the treatment of RA. According to preliminary studies, anakinra is suggested to be administered in combination with methotrexate which, again, may have severe side effects. However, all of the aforementioned agents show more or less severe side-effects.

Accordingly, there exists a need in the art for alternative and efficient treatments for autoimmune diseases. In particular there exists a continuing need in the art for means for prevention and/or an efficient treatment for inflammatory bowel diseases and rheumatoid arthritis.

Galectins are members of a highly conserved family of β-galactoside binding lectins that more and more emerge as significant regulators of immune cell homeostasis (1, 2). The biological properties of mammalian galectins include regulation of cell proliferation, inflammation, cell adhesion and cell death (3-5). Interestingly, despite extensive sequence homology and similar carbohydrate specificity, various members of this protein family behave as amplifiers of the inflammatory cascade, while others activate homeostatic signals to prevent immune responses (6). Hence, what applies to one member of the galectin-family, does not necessarily apply to other members as well.

Galectin-2 (Gal-2) was discovered during the cloning of galectin-1 and has 43% sequence identity to human galectin-1, revealing the greatest homology with galectin-1 among all others galectins examined (7). Galectin-2 is a non-covalent dimer with subunits of about 14 kDa (7). Galectin-2 is also known as beta galactoside binding lectin, Lectin I 14, LGALS2, Gal-2 or GAL-2. Expression of galectin-2 seems to be restricted to the gastrointestinal (GI) tract (8, 9). It is also known that intestinal epithelial cells that express galectin-2 do not normally express galectin-1 and that galectin-2 is not expressed at elevated levels in galectin-1 null mutant mice (10). Whereas the properties of galectin-1 have been studied more extensively, the function of galectin-2 has not been studied so far and thus remains unclear until now.

The ability of cells to adhere to constituents of the extracellular matrix (ECM) is a fundamental process involved not only in cell growth, apoptosis, angiogenesis, tumor invasion and inflammation (14, 15). Adhesion of T cells to the ECM is mediated by integrins, a large family of heterodimeric receptors consisting of eight different β subunits and 17 α chains that control matrix ligand specificity (16, 17). Galectins modulate cell adhesion by a different mechanism including bridging carbohydrate ligands on neighbouring cells, cross-linking galectins that are associated with the cell surface, direct binding to ECM compounds, protein-carbohydrate interactions or interaction with integrins (14, 18). The manifold options of galectins to influence cell-cell and cell-ECM adhesion may explain why galectins have pro- and anti-adhesive effects in different cell types and with distinct ECM constituents (6). Circulating blood T cells express only moderate amounts of β₁ integrins on their cell surface, yet these resting cells adhere poorly to ECM (17). Activation of blood T cells stimulates a rapid augmentation in β₁-mediated adhesion associated with enhanced integrin affinity and avidity (17), required for the migration of immune cells and horning to effector sites in the integrated human mucosal immune system (19). Galectin-1 binds to β1 integrin and transiently increases its availability on the cell surface whereas galectin-3 mediates the endocytosis of and thus down-regulation of β1 integrins in breast carcinoma cells (18, 20).

Immune cells must respond to antigens in a selective and balanced fashion that allows them to mount an effective response by progressing through the cell cycle, expanding and finally undergoing apoptosis once the antigen has been cleared (11, 12). Whereas this feature is usually tightly balanced by promoters and inhibitors of cell cycling and apoptosis (13), many diseases are based on either uncontrolled cell cycling or impaired apoptosis, leading to unrestrained cell proliferation and thus cancer or auto-immune disorders such as rheumatoid arthritis or inflammatory bowel diseases.

These diseases are up to date not curable, and existing treatments are either not very efficient or they show severe side effects.

Accordingly, it was an object of the present invention to provide for means for an efficient treatment and/or prevention of diseases with a malfunctioning immune system. It was furthermore an object of the present invention to provide for a treatment which shows fewer side effects than methods presently available. It was also an object to provide for means for an efficient treatment and/or prevention of diseases with an impaired apoptosis of T-cells.

All these objects are solved by the use of galectin-2 or of a nucleic acid coding for galectin-2 or of its complementary strand, or a nucleic acid hybridizing to such coding nucleic acid or its complementary strand, for the manufacture of a medicament for the treatment or prevention of a patient having a disease with impaired apoptosis of T-cells, macrophages and/or antigen-presenting cells, or for the manufacture of a medicament for the treatment or prevention of organ rejection in a patient having undergone organ transplantation, in particular solid organ transplantation.

In one embodiment said impaired apoptosis, in particular said impaired apoptosis of T-cells is involved in or associated with the pathogenesis of said disease.

In one embodiment said disease with impaired apoptosis of T-cells is selected from the group comprising autoimmune diseases and malignant T-cell diseases. Preferably said autoimmune diseases are selected from the group comprising rheumatoid arthritis, inflammatory bowel diseases, multiple sclerosis, psoriasis, lupus erythematodes, scleroderma, autoimmune hepatitis and autoimmune nephritis, wherein, more preferably, said inflammatory bowel diseases are Crohn's disease or colitis ulcerosa or indeterminate colitis.

Preferably said inflammatory bowel disease is Crohn's disease or colitis ulcerosa or indeterminate colitis.

In another embodiment, said autoimmune disease is rheumatoid arthritis.

In one embodiment said malignant T-cell diseases are selected from the group comprising peripheral and lymphoblastic/nodal and extranodal T-non-Hodgkin-lymphomas.

Preferably said galectin-2 is human or rat galectin-2.

In one embodiment said galectin-2 has an amino acid sequence selected from the group comprising SEQ ID NO:1 and SEQ ID NO: 2.

In one embodiment, said galectin-2 has more than 45%, preferably more than 60%, preferably more than 80%, more preferably more than 90% sequence identity to SEQ ID NO:1 or to SEQ ID NO:2.

In one embodiment, said galectin-2 comprises two monomers, which are preferably covalently linked to each other. In a preferred embodiment, said monomers are covalently linked to each other in the so-called dimerization domain of galectin-2. Preferably said covalent linkage is through a two-glycine linker. Such a covalent linkage is readily available to someone skilled in the art and has, for example, been described with respect to galectin-1 in Bättig et al., 2004, Molecular Immunology 41, 9-18, the disclosure of which is incorporated herein by reference in its entirety.

In one embodiment said galectin-2 is administered in combination with an agent suppressing T-cell proliferation and/or an agent inducing T-cell apoptosis.

Preferably said agent suppressing T-cell proliferation is selected from the group comprising steroids, macrolides, such as cyclosporin and rapamycin, tacrolimus, azathioprine, 6-mercaptopurine, methotrexate and cyclophosphamide.

Preferably said T-cell apoptosis inducing agent is selected from the group comprising anti-TNFα-antibody (infliximab, adalimumab and CDP 870), etanercept, leflunamide, natalizumab (anti-Integrin α4β7 mAb), visilizumab (anti-CD3 mAb).

In one embodiment said galectin-2 is administered in combination with a drug that induces T-cell apoptosis via a caspase-8 dependent pathway, wherein such drug may be one of the aforementioned ones.

In one embodiment said galectin-2 is administered in a patient failing or having failed to show a measurable response to a drug which is known to normally induce T-cell apoptosis via a caspase-8 dependent pathway, wherein such a drug may be one of the aforementioned ones.

In one embodiment said galectin-2 is administered in combination with anti-inflammatory drugs such as 5-Aminosalicylates (5-ASA), corticosteroids, mesalazine, olsalazin, balsalazin, sulfapyridin and non-steroidal anti-inflammatory agent and/or an antirheumatic agent, wherein, preferably, said antirheumatic agent is a disease modifying anti rheumatic drug (DMARD), wherein, more preferably, said disease modifying anti-rheumatic drug is selected from the group comprising aspirin, naproxen, diclofenac, ibuprofen, naprosyn, indomethacin, piroxican and biological drugs selected from the group comprising anakinra and etodolac.

In one embodiment, said antirheumatic agent is selected from the group comprising gold compounds, D-penicillamin, antimalaria drugs such as chloroquin, and sulfasalazine.

Preferably, said galectin-2 is administered in combination with cyclo-oxygenase-2-inhibitors (COX-2-inhibitors), wherein, preferably, said cyclo-oxygenase-2-inhibitors are selected from the group comprising celecoxib, rofecoxib, and valdecoxib.

In one embodiment, said galectin-2 is administered in combination with a T-cell activating agent.

In one embodiment, said galectin-2 is administered in combination with a β-galactoside, wherein, preferably, said β-galactoside is lactose.

In one embodiment said galectin-2 is administered in combination with or pre-processed with thiol-reducing or cystein-modifying reagent(s), preferably for maintenance of its activity.

In one embodiment, said galectin-2 is administered by systemical administration and/or topical administration, wherein, preferably, said administration occurs by ingestion, preferably orally or anally, and/or by injection, preferably by intravenous, intramuscular, intraperitoneal or subcutaneous injection, and/or by nasal application.

In one embodiment, said galectin-2 is administered as enema and/or as suppository and/or as delayed release dosage form, e.g. encapsulated in a pH dependent release matrix.

In one embodiment said galectin-2 is administered in a pegylated or non-pegylated form or as a mixture of the forms.

In one embodiment said galectin-2 is administered twice or more times per day or is continuously administered, e.g. by continuous infusion.

In one embodiment, said galectin-2 is administered daily in a dose range of 50 μg/kg body weight to 300 mg/kg body weight, preferably 1 mg/kg body weight to 100 mg/kg body weight. In a preferred embodiment, said galectin-2 is administered twice daily in an amount of 0.75 mg to 1.5 mg/kg body weight per dose. In a particularly preferred embodiment, said galectin-2 is administered twice daily in an amount of about 1 mg/kg body weight per dose.

In one embodiment, said patient is one having a pathological condition in which, before administration of galectin-2, a subset of the patient's T-cells and/or a subset of the patient's macrophages and/or a subset of the patient's antigen-presenting-cells fail to undergo apoptosis, preferably adequate apoptosis or wherein a subset of the patient's T-cells and/or macrophages and/or antigen-presenting cells show an impaired or defective apoptosis.

In one embodiment, said subset of T-cells are T-cells that have previously been activated, preferably via the CD3-pathway or the CD2-pathway or via mitogens, co-stimulatory molecules such as CD28 or CD40, or other pathways such as Toll-like receptors or integrins.

In one embodiment, said subset of T-cells and/or macrophages and/or antigen-presenting cells are not resting.

In one embodiment, said subset of T-cells and/or macrophages and/or antigen-presenting cells are not cells that have exited from the cell cycle or are not cells that are arrested in any phase of the cell-cycle.

Preferably said subset of T-cells and/or macrophages and/or antigen-presenting cells is primarily located in said patient's joints, preferably synovial joints, and/or in said patient's gastrointestinal tract, preferably the lining of said gastrointestinal tract, and/or in said patient's skin, and/or lung and/or liver and/or kidney and/or are a population of peripheral blood cells which are recruited in a mucosa during inflammation.

Preferably said patient is one having a pathological condition in which, before administration of galectin-2, the ratio between Bcl-2-protein and Bax-protein in T-cells is disbalanced in favour of the anti-apoptotic Bcl-2.

The objects of the present invention are furthermore solved by the use of galectin-2 or of a nucleic acid coding for galectin-2 or of its complementary strand, or of a nucleic acid hybriding to such coding nucleic acid or to its complementary strand, as an immunomodulating agent, wherein, preferably, galectin-2 acts on T-cells and/or macrophages and/or antigen-presenting cells, wherein, more preferably, said T-cells, macrophages and/or antigen-presenting cells are human.

As used herein, the term “galectin-2” is meant to designate a protein as encoded by SEQ ID NO: 1 or SEQ ID NO: 2 and proteins having more than 45%, preferably more than 60%, preferably more than 80%, more preferably more than 90% sequence identity thereto (i.e. over 45, 60, 80 or 90%, respectively, of the entire length of SEQ ID NO: 1 or SEQ ID NO: 2). It also is meant to signify any functional variant thereof which shows a selective induction of T-cell apoptosis in activated T-cells.

As used herein, the term “a disease with impaired apoptosis of T-cells” is meant to signify any disease which is accompanied by and/or caused by an impaired apoptosis of T-cells.

The term “involved in or associated with the pathogenesis of said disease”, as used herein is meant to signify that the impaired apoptosis may, but does not necessarily have to be the cause for said disease. In a preferred embodiment, said impaired apoptosis is one of the causes for said diseases, albeit not necessarily the only one.

The term “impaired apoptosis of T-cells” is meant to designate the notion that T-cells having served their function of eliminating an antigen, in the normal course of events (i.e. the non-pathological course of events) should undergo apoptosis, i.e. a programmed self destruction, whereas in a pathological condition, such as a disease, and where they show “impaired apoptosis” they do not undergo apoptosis at all or at a slower rate than normal.

The term “T-cell proliferation” is meant to signify the process in which T-cells undergo a cell-cycle of G1, S, G2 and M-phases, as a result of which the cell is divided into two cells at the end of the cycle.

The term “galectin-2 is administered . . . ”, as used herein, is meant to signify the it may be administered as a protein or as the nucleic acid encoding galectin-2-protein and/or as the nucleic acid strand complementary to the coding nucleic acid. In a preferred embodiment, it is meant to signify that galectin-2 be administered as a protein.

The term “ . . . is administered in combination with . . . ”, as used herein is meant to signify that galectin-2 may be administered in conjunction with other agents. The administration may be simultaneous or one after the other, it may be via the same route or via different routes, it may be in the same dosage form or via separate dosage forms.

An “agent suppressing T-cell proliferation” is an agent that locks (or slows down) a T-cell anywhere in the cell cycle.

An “agent inducing T-cell apoptosis” is meant to designate any agent that causes the cell to undergo programmed cell death, which is also commonly referred to as apoptosis.

The term “T-cell activating agent”, as used herein, is meant to indicate any agent capable of inducing a T-cell to enter from a resting state into a state in which the T-cell undergoes the cell cycle or at least parts of it.

Administration of galectin-2 may be in any form suitable for the recipient, provided that an uptake of galectin-2 into the recipient's organism is ensured. Administration may additionally be in combination with any pharmaceutically acceptable carrier. It may be systemic and/or topical, it may be injected, preferably intravenously, intramuscularly, intraperitoneally and/or subcutaneously, or it may be applied via other routes, e.g. as enema or suppository or via nasal application.

The inventors have surprisingly found that galectin-2 displays unique immunomodulatory properties characterized by specific binding of galectin-2 to the β1 subunit of integrins, a vigorous induction of T cell apoptosis by triggering the mitochondrial death pathway and alteration of integrin expression and cell adhesion, which makes galectin-2 particularly suitable for the treatment of diseases wherein T-cells are showing an impairment of their apoptotic behaviour. These diseases may be autoimmune diseases or malignant diseases, in particular malignant T-cell diseases. All peripheral and lymphoblastic/nodal and extranodal T-non-Hodgkin-Lymphomas show significant expression of β1 integrin chains. It can therefore be assumed that this increased surface accessibility of β1 integrin influences the behavior of the malignant cell. It is believed that Gal-2 down-regulates β1 accessibility in T-cells and may thus prevent attachment of malignant cells to the extracellular matrix. Therefore, Gal-2 may possibly have positive effects in the treatment of malignant T-cell diseases.

Galectin-2, in particular human galectin-2, is not immunogenic and therefore is particularly well suited for application as a medicament.

T cell function is determined by its activation and subsequent proliferation, cytokine secretion, but also termination of its action by apoptosis. Disturbances of this tightly balanced interplay are observed in many diseases, including inflammatory bowel disease (42). Galectin-1 has recently been shown to ameliorate experimentally induced colitis and hepatitis in mice by inducing T cell apoptosis (27, 43), whereas galectin-3 revealed anti-apoptotic properties in diverse cell populations (44). Thus, conclusions with respect to one galectin-family member cannot be transferred to another galectin-family member.

The expression, binding and biological function of galectin-2 was unclear until now, and the experiments described below show for the first time that galectin-2 has powerful immunomodulatory effects in human T cells, clearly distinct from other galectin family members.

In the following, the results of the experiments leading to the present invention are summarized, outlined and shortly discussed:

In contrast to galectin-1 and -3 (6), the present inventors found that galectin-2 is not expressed in resting or activated T cells, independent of the origin as naïve peripheral blood or tissue bound memory T cell. This finding is in accordance with the restricted galectin-2 mRNA expression found only in gastrointestinal epithelial cells (9). By immunohistochemistry the present inventors observed Gal-2 expression in colonic epithelial cells in specimen from patients with Crohn's disease and ulcerative colitis.

Galectins are carbohydrate-binding proteins that cross-link β-galactoside-containing cell surface glycoconjugates, resulting in the modulation of cell function (45). Our data show that galectin-2 binds to T cells within hours and remains bound for more than 72 hours. This binding was inhibited by lactose, indicating that galectin-carbohydrate interactions are responsible for galectin-2 binding to T cells. Comparable to galectin-1 (25), binding of galectin-2 to T cells was comparable in resting anti-CD3 or PMA/PHA (phorbol myristate acetate (PMA); phytohemagglutinin (PHA) stimulated T cells, demonstrating that galectin-2 binding to T cells is independent of their activation status.

The ability of galectin-2 to bind to T cells was significantly reduced by blocking integrin-β1 mAbs, identifying β1 as a candidate glycoprotein receptor that mediates binding of galectin-2 on the T cell surface. Galectin-1 interacts also with the β1 subunit of integrins (18), underscoring the important role of integrin-β1 in galectin binding. However, in contrast to galectin-1, galectin-2 binding to PBT could not be prevented by CD3 or CD7 mAb, indicating that both galectins have different binding sites on T cells (24). Immuno-precipitation using galectin-1 and galectin-2 coated magnetic beads confirmed the interaction of galectin-1 with the CD3, CD7 and β1 molecule (18, 24). In contrast with galectin-1, but in accordance with the blocking experiments, in which neither CD3 nor CD7 bound to galectin-2, the β1 subunit of integrins co-immunoprecipitated with galectin-2, indicating that the capability of β1 mAbs to inhibit galectin-2 binding to T cells is based on a physical interaction.

Apoptosis is a major immunomodulatory feature of T cells to terminate cell function once the antigen has been cleared, and thus preventing autoimmunity or malignancy. Furthermore, drugs that induce apoptosis of T cells or macrophages like anti-tumour necrosis factor-α, are strongly effective in treating autoimmune diseases, such as rheumatoid arthritis or inflammatory bowel disease (46, 47). Galectins studied so far have a remarkable capability to alter cell apoptosis in various cell types, e.g. galectin-1 induces and galectin-3 prevents cell death (6). However, nothing was known in this respect on galectin-2. Our data show that galectin-2 strongly induces T cell apoptosis to an extent even higher than that inducible by galectin-1. Galectin-2 required stimulation of the cells to induce apoptosis, showing that resting T cells are prevented from galectin-2 induced cell death. Galectin-2 also induced little amount of necrosis, demonstrating that galectin-2 not only induces the programmed cell death, but also kills cells by necrosis, a feature which has not previously been described for galectins at all.

The ability of lactose to suppress galectin-2 induced apoptosis indicates that β-galactoside binding activity is essentially necessary for galectin-2 induced cell death. Interestingly, since binding of galectin-2 to T cells is independent from the activation status of the cells as shown above, and since induction of apoptosis by galectin-2 requires additional cell activation, β-galactoside binding alone seems not to capable to initiate the cell death program. Galectin-2 binds to integrin β1 on the cell surface and β1 integrin mAb inhibit galectin-2 binding to T cells. However, the blocking β1 integrin mAb failed to inhibit galectin-2 induced apoptosis, suggesting that this receptor is not required for cell death. Cycloheximide was not able to pre-vent galectin-2 induced T cell death, indicating that in contrast to galectin-9 (28), de-novo protein synthesis seems not to be required, which was further substantiated by the fast induction of apoptosis in T cells following galectin-2 within 6-12 h. It has also been reported that galectin-1 induces apoptosis distinctively in CD4⁺ and CD8⁺ positive thymocytes (38, 48, 49) and that CD4⁺ human T cell lines are more susceptible towards galectin-9 induced cell death (28). However, when splenic T cell in nephritic rats were examined, galectin-9 induced selective apoptosis of activated CD8⁺, but not CD4⁺ T cells (50). When CD4 and CD8 positive T cell subpopulations were separately examined, the rate of galectin-2 induced cell death was comparable in each cell population, demonstrating that in PBT, galectin-2 induces apoptosis independent from their status as CD4⁺ or CD8⁺ T cell.

Apoptosis is executed by different pathways, initiated by distinct caspases. The present inventors' results demonstrate clear differences in caspase activity utilized by different galectins. When PBT were activated in the presence of galectin-2, caspase-3 and -9, but not -8 activity was induced. In addition, galectin-2 mediated apoptosis was prevented by the broad spectrum caspase inhibitor zVAD, as well as by caspase-3 and -9 inhibitors, further supporting the functional relevance of caspase-3 and -9 activation in galectin-2 induced cell death. Despite the fact that caspases are the central mediators of apoptosis (29), the role of caspases in galectin induced apoptosis has been rarely examined. It has been shown that galectin-1 induces caspase-8 and -9 activity in an experimental mouse model (27), but reduces caspase-3 activity in concanvalin A-induced hepatitis in mice (43). The ability of galectins to induce apoptosis is not restricted to prototype galectins since the tandem-repeat galectin-9 also induces apoptosis via a calcium-calpain caspase-1 dependent pathway (28). In contrast to galectin-2, also galectin-7 induced T cell apoptosis comparable to galectin-2, however, an entirely different pattern of caspases seemed to be involved. Whereas the broad spectrum caspase inhibitor zVAD and caspase-3 inhibitor zDEVD prevented galectin-7 induced apoptosis, blocking of the upstream caspases-1 and -8, but not -9 abridged T cell death. These findings demonstrate, that galectin-2 activates a distinct cascade of caspase to execute cell death compared to other galectins.

Caspase-9 is involved in the mitochondrial pathway of apoptosis and is activated when building a complex with cytochrome c and APAF-1 (29). Cytochrome c is released from mitochondria upon disturbance of the electrochemical gradient of the inner mitochondrial membrane, a process intimately regulated by bcl-2 family members (29, 51, 52). The present inventors' data show that galectin-2 disrupts the mitochondrial membrane potential and initiates cytochrome c release, showing that the mitochondrial pathway is used by galectin-2 to induce T cell death. Bcl-2 family members meet at the surface of mitochondria, and the susceptibility of a cell towards apoptotic stimuli is dependent on the relative balance of pro- and anti-apoptotic family members (52). Galectin-2 decreased anti-apoptotic bcl-2, while increasing pro-apoptotic bax levels, resulting in a lower bcl-2/bax ratio. In autoimmune diseases such as Crohn's disease, the bcl-2/bax ratio is higher than in controls (53), and the capability of galectin-2 to lower this ratio and thus to induce cell death in uncontrolled proliferating cells, may be an appealing approach to use galectin-2 therapeutically in such diseases.

Apoptosis and cell cycle are ultimately linked and T cells in G0 are protected from antigen-induced cell death (29, 54). The requirement to cycle is also true for galectin-2 induced cell death, however, in contrast to galectin-1, which profoundly inhibits T cell proliferation (38), galectin-2 did not modulate PBT cell cycling. The data so far available studying the impact of galectins on cell cycling mostly used thymidine incorporation to measure cell proliferation (38, 55). Although simple and reproducible, this method only measures DNA synthesis during the S phase, provides no information on the fraction of cells going through other phases and does not assess for apoptotic cells, unable to cycle. However, as determined by cyclin B1 expression, a highly sensitive tool to quantify progression through the cell cycle up to the G2/M-phase (56), galectin-1 and -7, but not galectin-2 strongly suppress cell cycle progression in PBT. Furthermore, demonstrating that the incapability of galectin-2 to influence T cell cycling is not due to a shift of cell cycle promoters and inhibitors, provides evidence that the cell cycle machinery of T cells is not affected by galectin-2, a feature clearly distinct from galectin-1 and -3. The present inventors and others have shown that galectin-1 binds to the T cell receptor (TCR) (24, 38) and Vespa and coworkers have demonstrated, that galectin-1 in conjunction with anti-TCR mAbs induces apoptosis, but antagonizes anti-TCR-induced IL-2 production (38). Our observation that galectin-2 does not bind to the TCR may explain, why galectin-2 does not inhibit T cell cycling, again demonstrating that the behaviour of galectin-2 is different to that of galectin-1.

T cells continually circulate in search for foreign antigens and the recruitment and retention of T lymphocytes within inflamed tissues are dependent on adhesion to an acellular network of proteins, glycoproteins, and proteoglycans known as the extracellular matrix (ECM) (57, 58). Adhesion is a critical component of the T cell mediated immune response and is therefore tightly regulated (59-61). Galectins modulate cell adhesion by influencing direct effects such as sterically preventing the adhesion to cell-surface integrins, but also by binding to extracellular domains of one or both subunits of an integrin or internalisation of integrins (20, 39, 45). Adhesion of T cells to ECM compounds is divergently regulated by galectins and depends on the cell type and ECM protein examined (6, 14, 62). Cell adhesion to ECM compounds is mediated by the β1 family of integrins, and the present inventors have shown that galectin-2 inhibits the conformational change of the integrin subunit required to achieve high affinity.

Furthermore galectin-1 inhibited T cell adhesion to both, collagen and fibronectin, whereas galectin-2 inhibited T cell adhesion to collagen type I, but in contrast to galectin-1, profoundly increased cell attachment to fibronectin. In contrast to both, galectin-1 and -2, galectin-7, did not influence T cell adhesion to collagen type I or fibronectin, indicating that galectin-2 exerts unique features not only with regard to apoptosis, but also cell adhesion. The attachment of T cells to their respective ECM compound was mediated by specific collagen and fibronectin receptors, as demonstrated by blocking experiments. It is not clear, why cell adhesion to fibronectin is promoted by galectin-2 despite the fact that galectin-2 reduces β1 integrin accessibility. However, regulation of cell adhesion by galectins seems to be complex, since galectin-1 has pro-adhesive properties in melanocytes, teratocarcinoma cells or fibroblasts, but inhibits T cell adhesion to laminin and fibronectin (reviewed in (6)). The same is true for galectin-3 that greatly increases the adhesion of neutrophils to various substrates (63), but blocks adhesion of tumour cell lines to laminin, fibronectin or collagen (64). Using a large panel of blocking integrin antibodies, the inventors demonstrated that binding of T cells was mediated through distinct collagen and fibronectin receptors, and pre-incubation of T cells with β1 mAbs further inhibited T cell binding to collagen and prevented the upregulation of cell adhesion to fibronectin by galectin-2. This suggests that modulation of cell adhesion by galectin-2 is β1 integrin dependent. Furthermore, the inhibition of cell adhesion by blocking β1 mAbs was prevented by galectin-2 pre-incubation, indicating that binding of galectin-2 to T cells can functionally inactivate the β1 subunit of integrins on the cell surface.

Regarding the potential for a clinical perspective it was further of interest to analyze whether and to what extent Gal-2 treatment might influence the balance of Th₁- vs. Th₂-derived cytokines. Monitoring the murine BI-141 hybridoma, Gal-1—in line with its CD3-binding capacity—impaired IL2-secretion, and murine LPS-treated spleen cells and human IL-2-activated T cells responded to Gal-1 treatment with reduced secretion of the proinflammatory Th₁-derived cytokines TNF-α and IF-γ (Rabinovic et al., 1999, Immunology, 97:100; Santucci et al., 2000, Hepatology 31:399). Gal-2 clearly shifted the secretion profile to Th₂-derived cytokines as shown in FIG. 9. The downregulation of secretion of TNF-α and IF-γ was accompanied by a marked increase of IL-5 secretion. These results broaden the knowledge on Gal-2-dependent effects and contribute to ascribe clinical relevance to the presented data. In transplantation models, the Th₁ cytokine profile often associates with allograft rejection, while the Th₂ profile favors the acquisition of tolerance. As Galectin-2 is suppressing TNF-α and IF-γ (Th₁ cytokines) and increases IL-5 as well IL-10 (Th₂ cytokines), Galectin-2 can be used as one or the sole component in preventing and/or treating organ rejection following organ transplantation, in particular solid organ transplantation.

In summary, the present inventors' studies provide clear evidence that galectin-2 binds in a carbohydrate-dependent fashion to the γ1 integrin subunit, but not the TCR complex. Galectin-2 induces T cell apoptosis via the mitochondrial, intrinsic death pathway, exclusively in proliferating, but not resting T cells. The induction of T cell apoptosis in activated cells, without down-regulating cell cycling, allows a sufficient T cell response after antigen contact, and prevents unselective apoptosis of resting T cells, not being involved in the course of the specific immune response. Furthermore, by selectively regulating cell adhesion to the ECM, galectin-2 may contribute to T cell homing once the cells are activated. Thus, the pre-sent inventors' data provide clear evidence that galectin-2 modulates the human immune system by mechanisms clearly distinct from other galectins, give a new insight how galectins may interact with T cells, and offers a new concept in the treatment of diseases with impaired T cell apoptosis like Crohn's disease or rheumatoid arthritis.

The invention will now be described by reference to the following specific description and the examples which are given to illustrate not to limit the present invention.

### IN THE FIGURES

FIG. 1 shows differential patterns of expression of galectins-1 and -2 in T cells. Gal-1 is consistently detected in resting and activated PBT on the level of mRNA (A) and protein (B), while Gal-2 expression was not detectable in PBT or LPT (A, B). Cells were cultured in the absence and presence of cross-linked anti-CD3 mAb for 72 h. mRNA expression was determined by PCR analysis and protein presence by Western blotting. The non-transformed intestinal epithelial cell line IEC-6 served as positive control. The graphs are representative of three individual series of experiments.

FIG. 2 shows binding of Gal-2 to T cells without and with stimulation. PBT were cultured in the presence of cross-linked anti-CD3 mAb or PMA/PHA for 24 h and biotinylated Gal-1 was used to monitor ligand presentation. An isotype control was added to exclude non-specific triggering and the haptenic sugar lactose was added to ascertain carbohydrate-dependent binding. Binding of Gal-2 to T cells was determined by two-color flow cytometry using streptavidin-labeled APC as marker. The graph is representative for three to five individual series of experiments.

FIG. 3 shows that galectin-2 binds to β1 integrin, but not CD3 or CD7. A) Cells were preincubated with CD3, CD7, and β1 mAb (1:100 dilution) for 1 h at RT and then cultured for additional 24 h in the presence of 5 μg/ml galectin-2 coupled to biotin. Cells were then harvested and binding of galectin-2 to T cells determined by two-color flow cytometry using streptavidin labelled-APC to detect biotinylated sites. B) Galectin-1 coimmunoprecipitates with CD3, CD7 and β1 integrin, whereas galectin-2 coimmunoprecipitate only with integrin β1. Cells were incubated with galectin-1 or -2 coated tosyl-activated beads and subsequently lysed. Bead-bound cell fragments were separated magnetically and immunoblotted for CD3, CD7 or β1. Both graphs are representative for four different experiments yielding similar results.

FIG. 4 shows induction of T cell apoptosis by galectin-2 and comparison to the respective activity of galectin-1. (A) Analysis of annexin-V and PI (propidium iodide) levels (annexin V binds to phosphatidylserine (PS). PS is flipped during early apoptosis from the inside to the outside of the cell membrane. Annexin-V detects extracellular PS and thus determines the number of a poptotic cells) revealed that galectin-2 induces more T cell apoptosis than Gal-1 and that cell activation was required to induce galectin-2 mediated cell death. Furthermore, Gal-2 shows very low necrosis effects in high concentrations. Cells were cultured with 0, 10, 25, 50, or 100 μg/ml galectin-1 or -2 in the presence or absence of cross-linked anti-CD3 mAb for 24 h. Apoptosis and necrosis were assessed by annexin-V and PI staining, respectively. Data represent mean ±SEM of six to seven independent experiments. *p<0.05 for increase vs. 0 μg/ml galectin; **p<0.05 for galectin-2 vs. galectin-1. (B). Cells were cultured with 50 μg/ml Gal-2 in the presence of cross-linked anti-CD3 mAb for the indicated periods of time. Extents of apoptosis and necrosis were assessed by annexin-V and PI staining, respectively. Data represent mean ±SEM of six to seven individual experiments. *p<0.05 for increase vs. Control (no presence of galectin); ⁺p<0.05 for effect of Gal-2 vs. that of Gal-1.

FIG. 5 shows that galectin-2 mediated apoptosis is caspase-3 and 9 dependent. A) Gal-2 induces activity of caspases-3 and -9, but not of caspase-8 (A). Cells were incubated in the presence or absence of 50 μg/ml Gal-2 for 24 h and caspase activity was assessed by flow cytofluorometry as described in Material and Methods. Illustrated plots are representative for four independent experiments which yielded very comparable results. B) The pan-caspase inhibitor zVAD and the caspase-3 inhibitor (zDEVD) impaired the activity of galectins-2, -7 and -1 to induce cell death. The caspase-1 inhibitor zYVAD blocked only Gal-7 induced cell death, whereas the caspase-9 inhibitor zLEHD blocked Gal-2- and -1-induced apoptosis. The caspase-8 inhibitor zLETD blocked Gal-7- and -1-induced cell death (B). Data represent mean ±SEM of four independent series of experiments. *p<0.05 for increase vs. no presence of galectin; ⁺p<0.05 for decrease vs. galectin.

FIG. 6 shows that galectin-2 disrupts the mitochondrial membrane potential, decreases bcl-2, but increases bax protein levels and induces cleavage of the DNA-fragmentation factor. Cells were cultured for 48 h in the presence of 0, 25 or 50 μg/ml galectin-2. A) Rhodamine 123 staining demonstrates a reduction of the mitochondrial membrane potential by galectin-2. B) Flow cytometry and C) western blotting show that galectin-2 profoundly reduces anti-apoptotic bcl-2, while increasing pro-apoptotic bax levels and inducing DFF-cleavage (DNA-fragmentation factor). Gel loading was controlled by monitoring a house-keeping gene product (GAPDH). All panels are representative of three different experiments.

FIG. 7 shows that galectin-2 does not alter T cell cycling. A) The number of cycling cells in S-, or G2/M-phase is comparable in the presence or absence of galectin-2. Cells were cultured with 0, 25 or 50 μg/ml galectin-2 and activated with cross-linked anti-CD3 monoclonal antibody for 72 h. Cell cycle phases were assessed by measuring DNA content by PI staining followed by flow cytometry. Each panel is representative of five different experiments. B) Cell cycle regulator expression is unchanged by galectin-2. Cells were cultured with 0, 25 or 50 μg/ml galectin-2 and activated with cross-linked anti-CD3 mAb for 72 h after which protein expression was assessed by western blotting. Each panel is representative of at least four different experiments.

FIG. 8 shows that galectin-1 and -7, but not -2 inhibit G2/M-cell cycle phase progression. Compared to galectin-2, flow cytometric analysis revealed that galectin-1 and -7 decrease cyclin B1 expression in the G2/M phase of activated PBT. Cells were cultured in the presence of cross-linked CD3 mAb for 72 h, after which cyclin B1 expression and DNA content were examined by flow cytometry. The figure is representative for four different experiments.

FIG. 9 shows that galectin-2 modulates profile of cytokine secretion by activated T cells. The effect of Gal-2 on cytokine secretion of resting and stimulated T cells was determined for γ-interferon (IFN-γ), tumor necrosis factor-α (TNF-α) and interleukins (IL)-10, -5, -4 and -2 by a commercially available cytofluorometric bead array using standards. Data represent mean ±SEM of three independent experiments. ⁺p<0.05 for data sets with stimulated T cells vs. resting T cells treated with 50 μg/ml Gal-2; *p<0.05 for data sets for Gal-2 treatment on stimulated T cells vs. T cells treated with CD3.

FIG. 10 shows that Galectin presence modulates cell surface presentation of integrins. Presence of galectins-1 and -2 (bold line) reduce cell surface presentation of integrin subunits β₁ and α₅ comparably, does not affect β₄ and slightly increases α₁ cell surface presence in PBT. In LPT, galectins-1 and -2 fail to modulate cell surface presence of β₁-integrin. The dotted line indicates staining by a isotype antibody as control. Cells were incubated in the presence or absence of 50 μg/ml galectins-1 or -2, respectively, and cell surface presence of integrins was assessed by flow cytometry. Negative control cells were gated to contain less than 3% positive cells. Representative histograms of three individual series of experiments are shown.

FIG. 11 shows that galectin-2 distinctively modulates T cell adhesion to collagen I and fibronectin. A) Galectin-1 inhibits cell adhesion to collagen type I and fibronectin, while galectin-2 inhibits PBT and LPT adhesion to collagen and increases T cell attachment to fibronectin. Galectin-7 does not significantly alter T cell adhesion to collagen or fibronectin. B) Specific blocking integrin mAb inhibit galectin-2 mediated modulation of cell adhesion to collagen type I and fibronectin. Pre-incubation of T cells with galectin-2 before adding integrin-β1 mAb reversed the inhibiting effect of the integrin β1 mAb. PBT and LPT were labelled with calcein and 5×10⁵ cells/well were allowed to adhere for 2 hrs to BSA-coated plastic as a control, collagen type 1, or fibronectin. Non-adherent cells were removed by washing. The fluorescence of adherent T cells was quantified using a fluorescence spectrophotometer. Data represent mean ±SEM of five independent experiments. *p<0.05 vs 0 μg/ml Gal-2; +p<0.05 vs 25 μg/ml Gal-2.

FIG. 12 shows the effect of varying concentrations of galectin-2 on T-cells from patients having RA or Crohn's disease and a control group. In detail, PBT from patients with CD and RA and healthy controls were stimulated with cross-linked anti-CD3 mAb antibodies for 48 hours in the presence and absence of the indicated concentration of human galectin-2. Apoptosis was determined by annexin-V staining.

FIG. 13 shows the effect of tacrolimus (Prograf® or FK506) on the apoptosis and necrosis of stimulated and unstimulated PBT. Cells were cultured with 0, 50, or 100 μg/ml tacrolimus in the presence or absence of cross-linked anti-CD3 mAb for 24 h. Tacrolimus is one of the most potent immunosuppressants and is currently being used for the prevention of organ rejection following solid organ transplantation and the treatment of inflammatory bowel diseases in selected patients. Yet it mainly induces a necrosis and no apoptosis of unstimulated as well as anti-CD3 stimulated PBT. In contrast thereto and as shown in FIG. 4A galectin-2 makes this distinction and it mainly induces apoptosis in PBT, thus indicating a better suitability of galectin-2 for the treatment of inflammatory bowel diseases.

FIG. 14 shows that galectin-2 prevents the development of dextran sodium sulfate induced colitis in mice. Colitis was induced in 8- to 10-week-old, female, Balb/c mice (Jackson Laboratories, Bar Harbor, Me.) by adding 5% DSS (Sigma Chemical Co., St. Louis, Mo.) (Dextran Sodiumsulfate (DSS) to their drinking water and allowing them to drink ad libitum. Groups of mice were treated either with Galectin-2 subcutaneously (s.c.) or intraperitoneally (i.p.) 2 h before DSS administration. In one trial the doses of Galectin-2 was doubled and compared to the treatment with FK506 (i.p.) in equal concentrations. Mice were weighed and inspected for diarrhea and rectal bleeding. 10 days after induction of colitis by DSS, mice were killed, their entire colon was resected, and its length was measured and weighed. The disease activity index (DAI; i.e., the combined score of weight loss and bleeding) was determined according to a well established standard scoring system.

FIG. 15A shows that galectin-2 reduces inflammation in a model of chronic colitis in mice. Colitis was induced in 30 8- to 10-week-old, female, Balb/c mice (Jackson Laboratories, Bar Harbor, Me.) by adding 5% DSS (Sigma Chemical Co., St. Louis, Mo.) (Dextran Sodiumsulfate (DSS) to their drinking water for the period of seven days and allowing them to drink ad libitum. At the 8^{th} day DSS administration was stopped for another seven days. This scheme of DSS administration for seven days followed by a period of seven days pure water administration was repeated. At the 29^{th} day 20 mice were treated with Galectin-2 intraperitoneally (i.p.) parallel to DSS administration. In one trial of this treatment a group of 10 mice were treated with 2 mg/kg Galectin-2 once a day and in another trial the same total dose was distributed in two administrations for a group of ten mice, e.g. 10 mice were treated with respectively 1 mg/kg Galectin-2 twice a day. Parallel a group of 10 mice were treated with 0,9% NaCl instead of Galectin-2 as control. At the 8^{th} day the Galectin-2 treatment was stopped. Parallel to the treatment mice were weighed and inspected for diarrhea and rectal bleeding. After stopping the treatment mice were killed, their entire colon was resected, and its length was measured and weighed. The disease activity index (DAI; i.e., the combined score of weight loss and bleeding) was determined according to a well established standard scoring system.

FIG. 15B shows the treatment scheme of the therapy trial used in FIG. 15A.

FIG. 16 shows the dependence of the disease activity index (DAI) on various treatments with galectin-2 and other drugs conventionally used for patients with severe IBD (inflammatory bowel disease) in mice with acute DSS colitis.

The disease activity index was scored in relation to various treatments and doses and dosage regimens. The drug galectin-2 human or rat) was administered once/twice or three times daily intraperitoneally or subcutaneously at 0.5 mg/kg body weight to 2 mg/kg body weight per dose.

FIG. 17 shows the dependence of the disease activity index (DAI) on various treatments with human galectin-2 in mice with chronic DSS colitis.

The disease activity index was scored in relation to various treatments and doses and dosage regimens. The drug galectin-2 (human) was administered once or twice daily intraperitoneally at 1 mg/kg body weight to 2 mg/kg body weight per dose.

FIG. 18 shows that galectin-2 results in a significant reduction of intestinal injury and inflammation in mice when compared to animals having received a physiological NaCl-solution as a control, as seen by tissue sections of the colon.

In control animals significant damage of the epithelial surface and a significant inflammatory infiltration of the mucosa can be observed, while in Galectin-2 treated animals only minor alterations of the mucosal surface and only few inflammatory cells within the mucosa can be identified.

FIGS. 19-22 show tissue sections of various organs of healthy mice that had been treated with high doses of galectin-2 to establish toxicity effects of supratherapeutic doses of galectin-2 in various organs (FIG. 19 kidney, FIG. 20 heart, FIG. 21 lung, FIG. 22 liver).

No evident toxic effects were observed macroscopically or by thorough histological evaluation in all of these organs.

### EXAMPLES

### Example 1

### Materials and Methods

### Reagents and Antibodies

CD3 mAb (mAb) (OKT3; Ortho Diagnostic System Inc., Raritan, N.J.), CD2 mAb (T11₂/T11₃; generously provided by Dr. Ellis Reinherz, Boston, Mass.), PMA (Sigma-Aldrich, St. Louis, Mo.) and PHA (Gibco, Grand Island, N.Y.) were used for T cell activation. The broad spectrum caspase inhibitor Z-Val-Ala-Asp(OMe)-fluoromethylketone (zVAD-fmk) was purchased from Biomol (Plymouth Meeting, Pa.), and caspase-1 inhibitor Z-Tyr-Val-Ala-Asp(Ome)-Ch₂F (Z-YVAD-fink), caspase-2 inhibitor Z-Val-Asp-Val-Ala-Asp(OMe)-fluoromethylketone (zVDVAD-fmk), caspase-3 inhibitor Z-Asp-Glu-Val-Asp(OMe)-fluoromethylketone (zDEVD-fmk), caspase-8 inhibitor Z-Ile-Glu-Thr-Asp(OMe)-fluoromethylketone (zLETD-fmk) and caspase-9 inhibitor Z-Leu-Glu(Ome)-His-Asp-(Ome)-Ch₂F (Z-LEHD-fmk) were purchased from Calbiochem (San Diego, Calif.). FITC-conjugated anti-cyclin B1 and PE-labelled anti-active caspase 3 were purchased from BD Pharmingen (San Diego, Calif.), CD3-PE, CD4-PE, CD8-FITC, CD3-PE, FITC- and PE-labelled polyclonal anti-mouse IgG were obtained from DAKO (Dako, Carpenteria, Calif.). Secondary FITC-labelled goat-anti mouse was purchased from Biosource (Camarillo, Calif.) and APC-labelled streptavidine was obtained from Caltag (Burlingame, Calif.). The Carboxyfluorescein (FAM) caspase detection kits, measuring caspase activity, were obtained from Biocarta (San Diego, Calif.). Lactose, sucrose, cyclophosphamide, and rhodamine 123 were purchased from Sigma-Aldrich. Propidium iodide (PI) was purchased from Calbiochem (San Diego, Calif.). All protease- and phosphatase inhibitors used for western blotting were purchased from Sigma-Aldrich. The antibodies against human caspase 3, DFF, PARP, retinoblastoma (Rb) protein, cyclin A, p21, p27, and p53 were purchased from BD Pharmingen. Antibodies against human bax, bcl-2, and cytochrome c were obtained from Santa Cruz Biotechnologies (Santa Cruz, Calif.). The cytometric bead array kit was purchased from BD Pharmingen, and IFN-γ, IL-10, and IL-2 ELISA kits were obtained from R&D (Minneapolis, Minn.). Galectin-2 and -7, as well as Galectin primers were kindly provided by H. -J. Gabius (Institute of Physiological Chemistry, Faculty of Veterinary Medicine, Munich, Germany). Furthermore, isolation and Expression of Galectin-2 is described in (65). Or Galectin-2 can be purchased at R&D Systems Inc., Minneapolis, USA, catalogue No. 1153-GA-050 or 1153-GA-050/CF. For flow cytometry and blocking experiments, anti-human α1 integrin mAb (clone FB12), anti-human integrin α2 mAb (clone P1E6), anti-human integrin α3 mAb (clone P1B5), anti-human integrin α4 (clone P1H4), anti-human integrin α5 (clone P1D6), anti-human integrin β2 (clone P4C10) and β2 (clone 3E1) were used (all from Chemicon, Temecula, Calif.).

### Preparation of Galectins

cDNAs for human and rat galectins-2 were cloned from mRNA pools of HT-29 colon carcinoma cells or rat duodenum, respectively. The introduction of a NcoI restriction site resulted in a Thr(Ser)2Ala substitution in the corresponding protein sequences. For recombinant expression the pQE-60 vector system (Qiagen, Hilden, Germany) was used and lectin purification was performed with affinity chromatography on lactosylated Sepharose 4B, obtained by ligand coupling after divinyl sulfone activation, as crucial step (Gabius, 1990, Anal. Biochem., 189:91; André et al., 1999, J. Cancer Res. Clin. Oncol. 125:461). Homogeneity and quaternary structure were ascertained by gel filtration and one- and two-dimensional gel electrophoresis. Galectins-1 and -7 were prepared as described previously, and the galectins were biotinylated under activity-preserving conditions by an optimized procedure, label incorporation quantitated by two-dimensional gel electrophoresis and maintenance of carbohydrate-binding activity ascertained by solid-phase assays (Andre et al. 2001, ChemBioChem, 2:822; Purkrabkova et al., 2003, Biol. Cell 95:535). Polyclonal antibodies were raised in rabbits, and cross-reactivity to related galectins was excluded by ELISA assays and Western blotting (Kaltner et al., 2002, Cell Tissue Res. 307:35; Nagy et al., 2003, Cancer 97:1849).

### Preparation of T Lymphocytes

PBMC (peripheral blood mononuclear cells) from healthy volunteers were isolated from heparinized venous blood using Ficoll-Hypaque density gradients. For isolation of peripheral blood T lymphocytes (PBT), PBMC cells were incubated for 30 minutes at 4° C. with magnetically-labelled CD19, CD14, and CD16 Ab directed against B-lymphocytes, monocytes, and neutrophils, respectively (Miltenyi Biotec Inc., Bergisch-Gladbach, Germany). T cells were then collected using a magnetic cell sorting system (MACS, Miltenyi Biotec Inc.). Lamina propria T cells (LPT) were isolated from surgical specimen obtained from patients admitted for bowel resection for malignant and non-malignant conditions of the large bowel, including colon cancer and benign polyps as previously described (21). Briefly, the dissected intestinal mucosa was freed of mucus and epithelial cells in sequential washing steps with DTT and EDTA, and digested overnight at 37° C. with collagenase and DNase. Mononuclear cells were separated from the crude cell suspension by layering on a Ficoll-Hypaque density gradient. For LPT purification, macrophage-depleted lamina propria mononuclear cells were incubated for 30 minutes at 4° C. with magnetically-labelled beads as described above and collected by negative selection using the MACS system. As assessed by flow cytometry, the purified PBT and LPT populations contained >99% and >92% CD3⁺ cells, respectively.

### PCR

Total RNA was isolated using the Advantage RT-for-PCR-kit (Clontech, Palo Alto, Calif.) according to the manufactor's instruction. Two point five μg aliquots of total RNA were reverse transcribed essentially as previously described (8) and Galectin-2 mRNA amplified using the following primers: sense, 5′-ATGACGGGGGAACTTGAGGTT-3′ and anti-sense 5′-TTACGCTCAGGTAGCTCAGGT-3′. The thermal cycle commenced with a hot start 94° C. for 4 minutes followed by 36 cycles each consisting of 94° C. for 60 seconds, annealing for 60 seconds at 60° C., extension at 72° C. for 120 seconds, and terminated after a final 10 minute period at 72° C. The products were separated on a 1% TAE agarose gel and visualized by ethidium bromide staining under UV light.

### Western Blotting

For immunoblotting cells were washed in PBS, and lysed in cell lysis buffer (1% Triton-X, 0.5% NP-40, 0.1% SDS, 0.5% sodium deoxycholate, 5 mM EDTA, 50 mM and 50 mM protease- and phosphatase-inhibitor cocktail 2, 1 mM PMSF, 100 μg/ml trypsin-chymotrypsin inhibitor, 100 μg/ml chymostatin in PBS). The concentration of proteins in each lysate was measured using the Bio-Rad protein assay (Bio-Rad Laboratories, Hercules, Calif.). Equivalent amounts of protein (10 μg) were fractionated on a 10-20% Tris-glycine gel and electrotransferred to a nitrocellulose membrane (Novex, San Diego, Calif.). Membranes were blocked with 5% milk in 0.1% Tween 20-PBS (Fisher Scientific, Hanover Park, Ill.), followed by incubation for 60 m in at room temperature with the indicated primary antibody. The membranes were washed six times with 0.1% Tween 20-PBS and then incubated for 1 h with the appropriate horseradish peroxidase-conjugated secondary antibody (Santa Cruz Biotechnology), washed, and incubated with the chemiluminescent substrate (Perkin-Elmer, Carlsbad, Calif.) for 5 minutes. The membranes were then exposed to film (Amersham, Arlington Heights, Ill.).

### Determination of Binding of Biotinylated Gal-2

To measure Gal-2 binding to T lymphocytes, aliquots of cell suspensions were kept as controls or stimulated for 1, 6, 12, 24, 48, and 72 h with anti-CD3 mAb or PMA and PHA and incubated in the presence of 5 μg/ml biotin-labeled Gal-2. Cells were then harvested, washed and stained with anti-CD3-FITC mAb and APC-labeled streptavidin (Biosource) followed by flow cytofluorometric analysis. Cells cultured in the absence of biotin-labeled Gal-2 and stained with FITC-labeled mouse IgG (BD Pharmingen) or APC-labeled streptavidin served as isotype controls. Each analysis was performed on at least 20,000 events.

### Magnetic Separation of Galectin-2 Containing Complexes

For separation of galectin-2-associated complexes, 1×10⁸ tosylactivated, supermagnetic, polysterene coated beads (Dynalbeads, Dynal Biotech, Oslo, Norway) were coated either with 350 μg BSA, Galectin-1, Galectin-2, CD7, integrin-β1 or OKT3 overnight at 37° C. using tilt rotation. After incubation, the beads were washed in 0.2 M Tris (pH 8.5 containing 0.1% BSA), and then incubated with 2×10⁶ PBT for 1 h at 37° C. Unconjugated cells were removed by extensive washing and cells were subsequently lysed with homogenization buffer (20 mM Tris HCl, pH 7.6; 10 mM MgCl₂, 0.05% Triton-X 100, 50 mM phosphatase- and 50 mM protease inhibitor cocktail), resuspended in SDS-DTT protein loading buffer, heated to 95° C. for 5 min and submitted to SDS-PAGE electrophoresis.

### Adhesion Assay

96 well flat-bottom plates were precoated overnight at 4° C. with 12 μg/well fibronectin (Chemicon) in DPBS, 40 μg/well collagen type I (Sigma-Aldrich) in 0.1 M acetic acid, or 3% BSA (Sigma-Aldrich) in DPBS as a control and then washed three times with DPBS. Freshly isolated T cells were fluorescently-labelled with 5 μM calcein-AM (Molecular Probes, Eugene, Oreg.) for 30 min at 37° C. in 5% CO₂ at 5×10⁶ cells/ml in RPMI. The cells were then washed three times with RPMI containing 5% FCS. Calcein-labelled T cells were resuspended in RPMI and 5×10⁵ T cells/well were added to 96-well plates containing purified ECM components or BSA for the indicated times. Saturating concentrations of integrin-blocking mAbs (predetermined by flow cytometry) were preincubated with T cells for 30 min at 37° C. before adding T cells to the wells for the adhesion assay. Nonadherent cells were removed by a standardized washing technique that was developed to minimize background binding, which includes both an orbital and a rocking motion repeated three times with RPMI. Adhesion was quantified with a multiwell fluorescent spectrophotometer (Tecan, Groedig, Austria). For each experimental group the results were expressed as the mean percentage±SD of bound T cells from triplicate wells.

### Determination of Extent of Apoptosis and Necrosis

Apoptosis was determined by monitoring fragmentation of nuclear DNA and access of annexin V to c ell surface phosphatidylserine (PS). To detect fragmentation of nuclear DNA, T cells were cultured in the presence and absence of the respective galectins, with or without blocking antibodies or further activation for 48 h. Thereafter, cells were fixed in 0.2 ml of 37% formaldehyde for 10 min at room temperature. The cells were then treated with 1 ml PBS containing 0.2% Nonidet P-40 (Sigma-Aldrich) for 2 min at room temperature and then rinsed once in PBS. The apoptotic cells were detected by staining of nuclear DNA with 4,6-diamino-2-phenylindole (DAPI) (Calbiochem). DAPI was added at a concentration of 0.2 ng/ml of PBS and cells were incubated at room temperature for 20 min in this solution. Cells were rinsed twice in PBS and the coverslips were maintained cell side down on a microscope slide and analyzed using a Zeiss Axiovert 135M microscope (Carl Zeiss, Oberkochen, Germany). To detect early phases of apoptosis, access to PS was measured. Cells were cultured as described, harvested at the respective time points, stained with FITC-labeled annexin-V and PI and analyzed by flow cytometry using the CellQuest software program (BD Pharmingen). A minimum of 15,000 cells was monitored in each case.

### Assessment of Mitochondrial Membrane Potential

Rhodamine 123 is a fluorescent cationic dye that accumulates in the mitochondrial matrix because of its charge and solubility in both the inner mitochondrial membrane and matrix space (22). Based on the observation that accumulation of this lipophilic dye is in proportion to ΔΨ, and that deenergizing of the mitochondria decreases rhodamine 123 fluorescence (23), the inventors measured the mitochondrial membrane potential of PBT. Cells were stimulated in the presence or absence of galectin-2 with CD3 for 24 h, harvested, washed and resuspended in 1 ml of rhodamine 123 (10 μg/ml, Sigma-Aldrich) for 30 min at 37° C. in the dark. The samples were washed twice in cold PBS and fluorescence analysis by flow cytometry using an argon ion laser with an emission filter at 530 nm was performed immediately without fixation (BD FacsCalibur). PBT without stimulation and unstained samples were used as controls.

### Flow Cytometric Analyses

For surface staining, cells were washed twice in ice-cold PBS and 1×10⁶ cells were resuspended in flow buffer (HBSS containing 1% BSA and 0.1% sodium azide). Cells were incubated with the respective mAb at predetermined saturating concentrations or with isotype-matched nonspecific mouse mAb (DAKO) for 30 min at 4° C., washed twice with flow buffer, and incubated with FITC-conjugated goat anti-mouse IgG for 30 min at 4° C. The cells were again washed twice with flow buffer, fixed in 1% paraformaldehyde and analyzed by a single laser flow cytometer (FacsCalibur, Beckman Coulter), using Cell Quest software (BD Pharmingen). Cells stained with a negative control Ab were gated to contain <2% positive cells. To perform analysis of intracellular proteins, cells were washed twice with PBS, adjusted to 1×10⁶ cells per sample, and fixed in 90% methanol at −20° C. After fixation, cells were washed twice with PBS and incubated with a monoclonal mouse anti-bax, Bcl-2, Rb, Cyclin A, p21, p27, or p53 antibody for 45 minutes at 4° C., followed by an incubation with a goat anti-mouse FITC-conjugated mAb (Biosource) for 45 minutes at 4° C. Thereafter cells were washed and analyzed by flow cytometry as described above. The background level of immunofluorescence was determined by incubating cells with FITC-conjugated mouse IgG (isotype control).

### Determination of Caspase Activity

To measure caspase activity, 5×10⁵ PBT were incubated in the presence or absence of 50 μg/ml galectin-2 together with derivates of the PE-labelled affinity purified anti-caspase-3 Ab (BD Pharmingen), the carboxyfluorescein-labelled caspase-8 inhibitor FAM-LETD-fmk or caspase-9 inhibitor FAM-LEHD-fmk (both from Biocharta, Hamburg, Germany), all binding irreversibly to activated caspase-3, -8 or -9, respectively. Cells were than analyzed by flow cytometry (FACS-Calibur, BD) and the increase of caspase activity determined after proper gating in correlation to untreated cells.

### Analysis of T Cell Cycling

Flow cytometry was performed after staining for DNA content, and cyclin B1, essentially as previously described (21). Briefly, cells were washed twice with PBS, adjusted to 1×10⁶ cells/sample and fixed in 90% Methanol at −20° C. After fixation, cells were washed twice with PBS, incubated for 45 min at 4° C. with a cyclin B1-FITC conjugated monoclonal antibody. After the final wash, cells were resupended in PBS and 5 μl of RNase (0.6 μg/ml, 30-60 Kunitz Units, Sigma), incubated at 37° C. for 15 min and the chilled on ice. One hundred and twenty five microliters of PI (200 μg/ml) were added prior to analysis by flow cytometry. Each analysis was performed on at least 25.000 events.

### Measurement of Cytokine Secretion

To determine cytokine secretion, PBT were cultured for 48 h with or without anti-CD3 mAb and incubated in the presence or absence of 0, 10, or 50 μg/ml Gal-2. The supernatant was then collected and cytokine secretion determined by a cytometric bead array, performed according to the manufacturer's instruction (BD Pharmingen). Briefly, six bead populations with distinct fluorescence intensities, coated with capture antibodies specific for TNF-α, IFN-γ, IL-10, IL-5, IL-4, and IL-2 proteins, were mixed with PE-conjugated detection anti-bodies and incubated with recombinant standards or test sample to form sandwich complexes. Following acquisition of sample data using flow cytometry, the cytokine concentrations were calculated using the BD CBA analysis software.

### Statistical Analysis

Statistical analysis was performed using the paired student's t test. Results are expressed as mean ±SEM, and significance was inferred with p values<0.05.

### Example 2

### Immunological Effects of Galectin-2

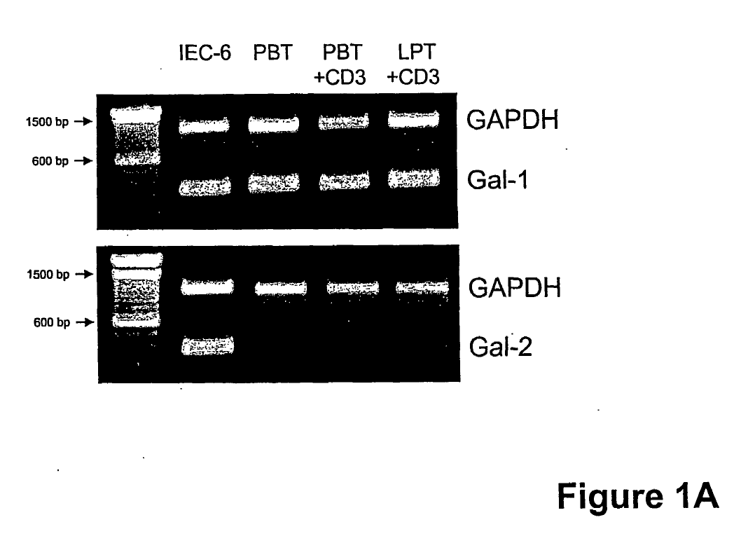

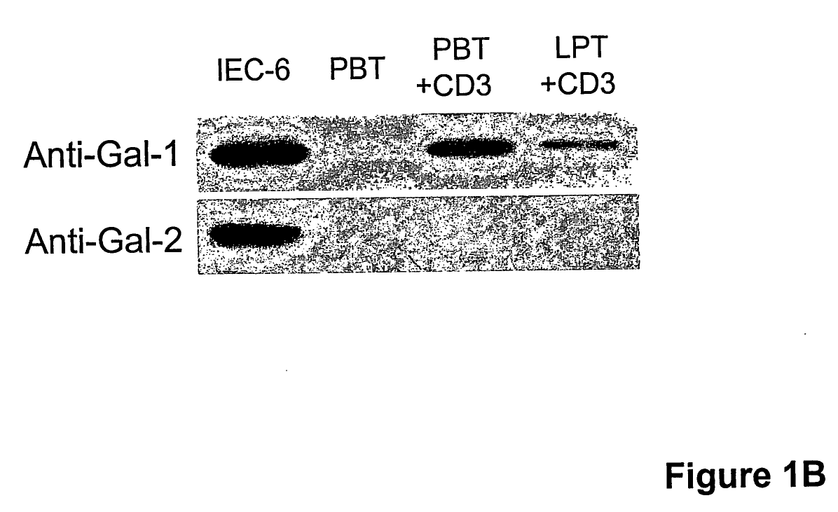

### Expression of Galectin-2 in T Cells

Whereas galectin-1 and -3 are widely distributed among various mammalian tissues and cell types, other galectins such as galectin-4 and -6 are expressed in a more tissue restricted fashion (6-8). The expression of galectin-2 is different from other galectins and seems to be restricted to the GI tract (8, 9). Within the GI tract, galectin-2 mRNA expression was detected in the duodenum, jejunum, and to a lesser extent in the coecum, colon and rectum (9), however protein expression in human tissues has not yet been examined. Since galectins exert strong immunoregulatory effects, the inventors first wanted to know, if galectin-2 is expressed in resting and activated peripheral and intestinal T cells. PCR analysis demonstrated, that in contrast to galectin-1, galectin-2 mRNA is not expressed in resting or activated PBT (FIG. 1A). Consequently, western blot analysis failed to detect protein expression of galectin-2 in resting or stimulated PBT or LPT (FIG. 1B). As positive control the rat non-transformed intestinal epithelial cell line IEC-6 was used, demonstrating galectin-2 expression in these cell lines (FIG. 1B).

### Carbohydrate-Dependent Binding of Gal-2 to T Cells

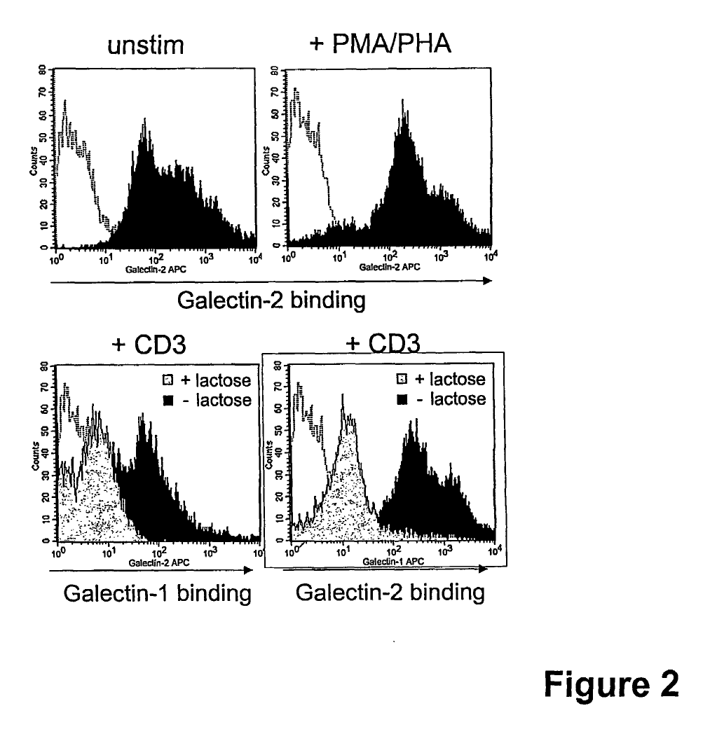

For this purpose, the present inventors biotinylated the lectin under activity-preserving conditions and verified maintenance of the carbohydrate-binding specifity by solid-phase assays (not shown). Labeled Gal-2 bound to unstimulated T cells in a β-galactoside-specific manner, because presence of 50 mM lactose as pan-galectin inhibitor reduced binding by more than 70% (FIG. 2). Sucrose tested at the same concentration failed to affect the staining intensity in flow cytometry, excluding non-specific effects (not shown). To figure out whether and how stimulation of T cells might modify reactivity of Gal-2, the present inventors used either PMA/PHA or anti-CD3 antibody. In both cases, they found an increase in cell staining (FIG. 2). Evidently, Gal-2 which is not expressed in T lymphocytes can bind to carbohydrate ligand(s) on the T cell surface. The extent of binding is increased by cell stimulation. Compared to Gal-1, binding of Gal-2 appeared to be slightly stronger. To gain insight into the biochemical nature of cell surface targets for Gal-2, the present inventors employed the antibody-blocking approach targeting defined cell surface glycoproteins and used Gal-1 as internal control.

### Gal-2 Associates with β₁-Integrin But not CD3 or CD7

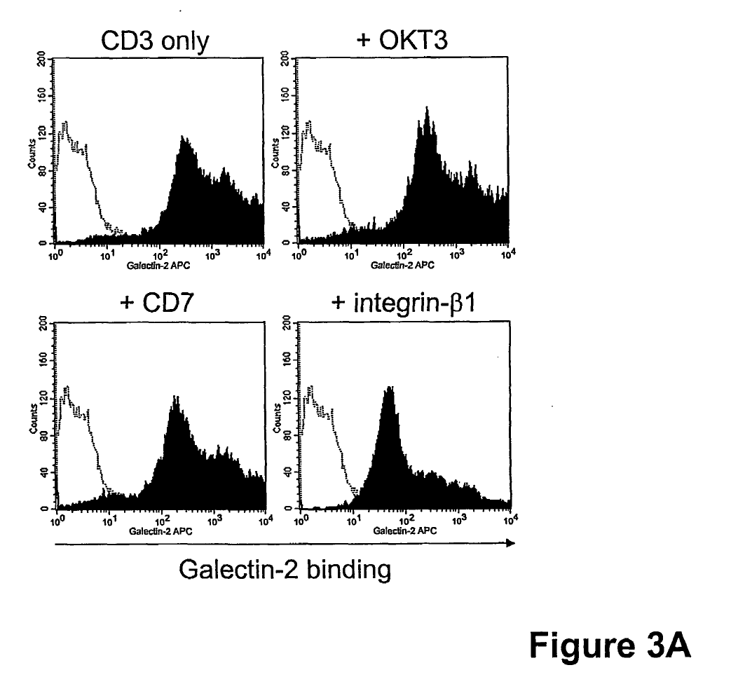

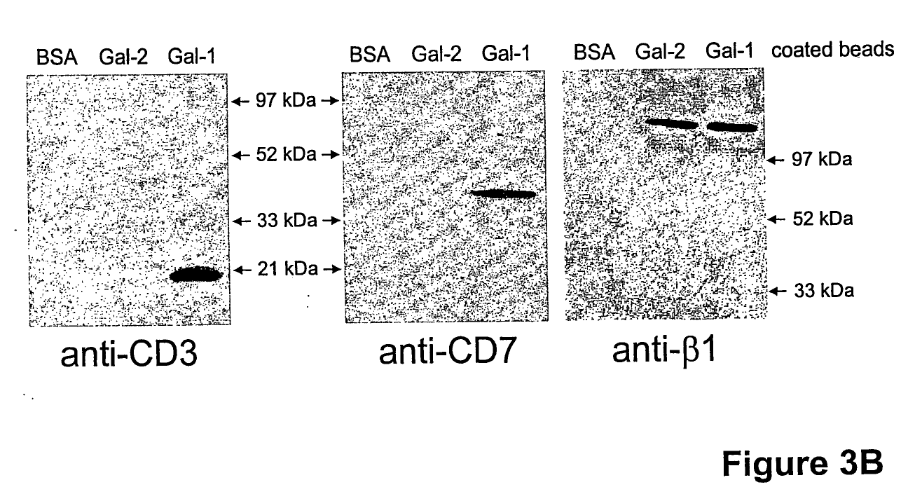

Any interference on galectin binding to stimulated T cells by the presence of epitope-specific antibodies was quantitated by flow cytometry. Cell surface binding of Gal-1 was expectably reduced by antibodies against CD3, CD7 and β₁-integrin, respectively, intimating an interaction with the antigen concerned or a determinant in close vicinity (not shown). Gal-2 binding, however, was not significantly influenced by the presence of reagents specific for CD3 and CD7 (FIG. 3A). Despite a high sequence alignment score galectins-1 and -2 actually have distinct preferences for glycoproteins as ligands. In contrast, the antibody against β₁-integrin was a potent inhibitor, markedly reducing cell staining (FIG. 3A). As control, the antibody against β₂-integrin failed to inhibit Gal-2 binding to T cells (not shown). Relative to presence of lactose the comparison of the two profiles intimates that β₁-integrin appreciably contributes to the overall ligand panel. Thus, theses experiments add a clear difference in ligand selection to the differences in the expression profile. In addition to the blocking approach, the present inventors ran Western blotting on the fraction of extract glycoproteins with ligand capacity for galectins. In the case of Gal-1, presence of CD3, CD7 and β₁-integrin was predicted by the cytofluorometric results. Indeed, these glycoproteins could be detected after an affinity fractionation of extract with immobilized Gal-1 (FIG. 3B). Remarkably, a complete correlation for cytofluorometric and biochemical results was also found for Gal-2: no trace of CD3 or CD7 but the presence of β₁-integrin could be visualized by the Western blots in the fraction of Gal-2-binding glycoproteins (FIG. 3B). A BSA control ascertained the absence of non-specific protein-protein interactions (not shown). Despite their conspicuous structural relationship the two galectins bind distinct glycoproteins, and Gal-2 homes in on β₁-integrin or a tightly associated glycoprotein as a major target. Having described the cell-binding capacity of Gal-2 and detected absence of reactivity to CD3 and CD7 by two independent approaches, the inventors next asked the question as to whether cell binding might induce apoptosis.

### Induction of T Cell Apoptosis by Galectin-2

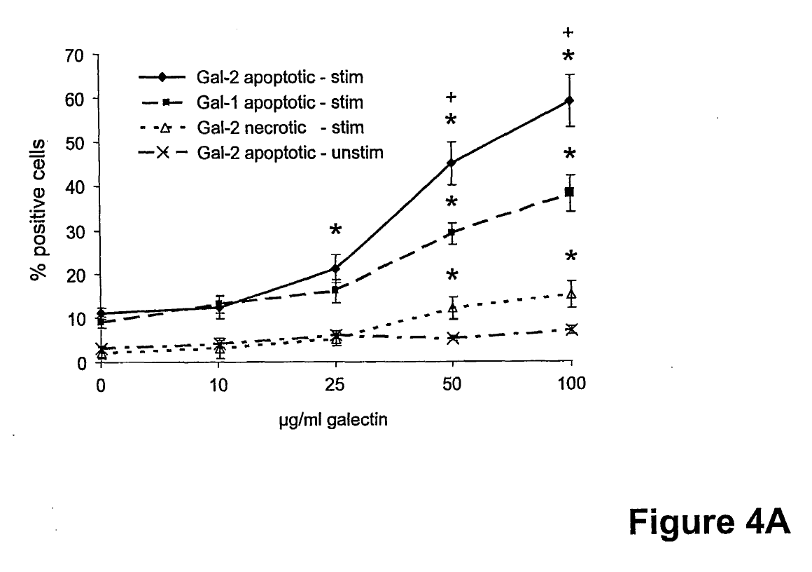

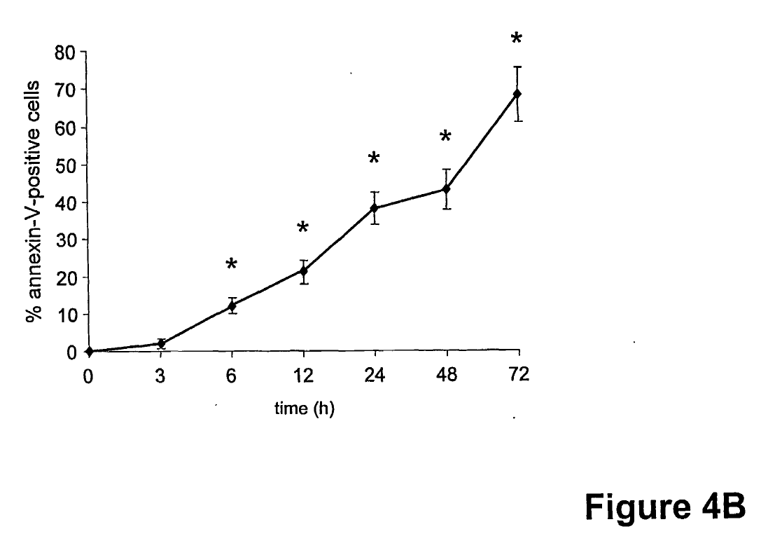

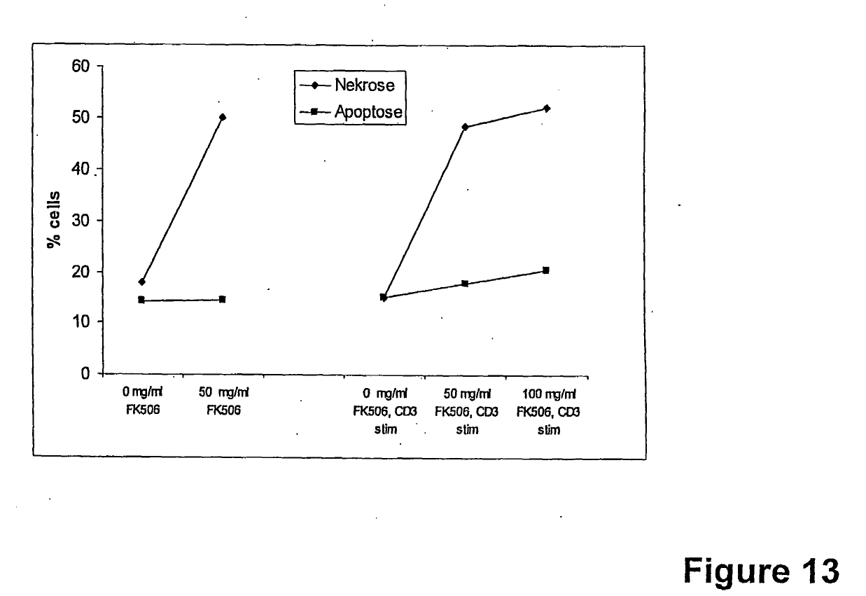

Galectins have a variety of modulatory effects in T cells and can induce, but also inhibit T cell apoptosis (25, 26). Since it is not known if galectin-2 modulates T cell apoptosis, the inventors first assessed if galectin-2 induces surface exposure of phospatidylserine, as measured by annexin-V staining, in activated and non-activated PBT. The time course of annexin-V staining in the presence of Gal-2 followed a gradual increase over 72 h (FIG. 4B). When PBT were cultured in the presence of 0, 10, 25, 50 and 100 μg/ml galectin-2, but without cell stimulation, galectin-2 failed to induce T cells apoptosis (FIG. 4A). In contrast, when PBT were activated by anti-CD3 mAbs, galectin-2 induced a dose-dependent apoptosis, but also, albeit to a much lesser extent, necrosis, of T cells (FIG. 4A). In contrast thereto, the immunosuppressant tacrolimus (FK506), which is commonly used to prevent organ rejection following allogenic transplantation of kidneys and livers and to treat refractory inflammatory bowel diseases in selected patients, makes no distinction between stimulated and unstimulated T-cells and mainly induces necrosis (FIG. 13) thus causing possibly septic effects. The percentage of T cell apoptosis induced by galectin-2 was higher than that induced by galectin-1 (FIG. 4A), and was comparable in CD4 and CD8 positive T cell populations (data not shown). To examine the requirement of de-novo protein synthesis for galectin-2 induced cell death, cells were exposed to 0, 10, and 1000 nM of cyclophosphamide, a protein synthesis inhibitor (see Kashio, Y., K. Nakamura, M. J. Abedin, M. Seki, N. Nishi, N. Yoshida, T. Nakamura, and M. Hirashima. 2003. Galectin-9 induces apoptosis through the calcium-calpain-caspase-1 pathway. J. Immunol. 170:3631), in the presence and absence of galectin-2. Furthermore, the inventors examined the effect of lactose on the ability of galectin-2 to induce apoptosis. Whereas cyclophosphamide failed to modulate galectin-2 induced T cell death (data not shown), apoptosis was largely inhibited by 50 mM lactose, but not sucrose.

### Galectin-2 Mediated Apoptosis is Caspase-3 and -9 Dependent

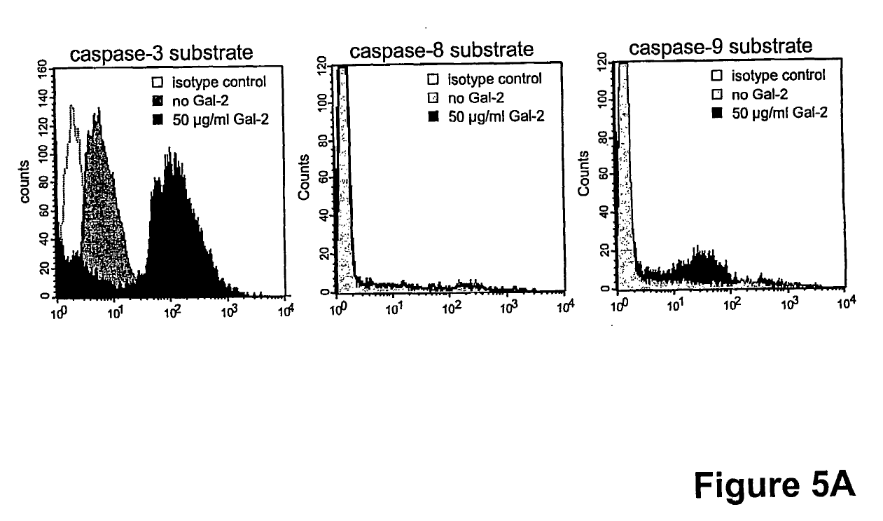

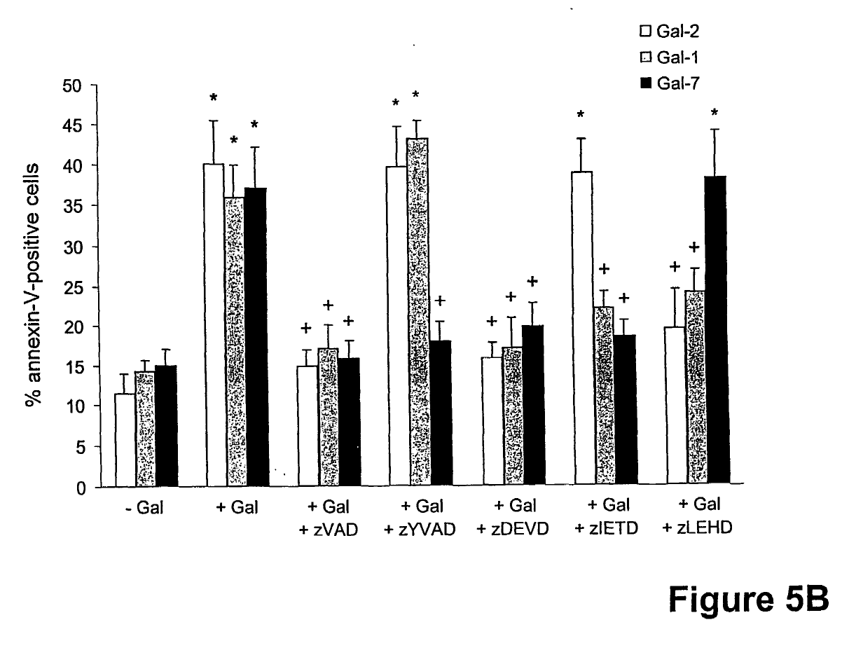

Having established that galectin-2 induces T cell apoptosis, the inventors were interested to assess the apoptotic pathways used by galectin-2. They therefore measured caspase activities in galectin-2 treated, anti-CD3 activated PBT using fluorogenic substrate assays. When PBT were activated by the CD3 pathway, galectin-2 induced a significant increase in caspase-3 and -9, but not -8 activity (FIG. 5A). To assess the functional relevance of this finding, the inventors evaluated the effect of caspase inhibition on galectin-2 induced T cell apoptosis. Both specific and broad spectrum caspase inhibitors were tested, and all contained fluoromethyl-ketone (fmk), which renders the inhibitory effect irreversible (all used at 50 μM). Caspase-1 (zYVAD) and -8 (zIETD) inhibition had no effect on galectin-2 induced T cell apoptosis. In contrast, caspase-3 (zDEVD), -9 (zLEHD) and the broad spectrum caspase inhibitor (zVAD) significantly inhibited galectin-2 induced cell death (FIG. 5B). Since it has been shown that monomeric galectin-1 increases both, caspase-8 and -9 activity (27), and the tandem-repeat-type galectin-9 induces apoptosis via caspase-1, but not -8 or -9 (28), the inventors also assessed the effect of galectin-7, like galectin-2 a monomeric prototype galectin (2), on T cell apoptosis. Although galectin-7 induced PBT apoptosis comparable to galectin-2, preincubation with zVAD, zDEVD, and zIETD, but not zLEHD inhibited galectin-7 mediated cell death, indicating that galectin-7 mediates T cell apoptosis via caspase-3 and -8, but not -9 (FIG. 5B). Having documented the pattern of caspase activation by Gal-2, the inventors proceeded to include Gal-1 in aliquots of their cell preparations comparatively. The ability of zVAD to reduce Gal-1-induced cell death provided evidence that caspases are involved in Gal-1-mediated apoptosis (FIG. 5B). A conspicuous difference was the marked effect of the caspase-8 inhibitor (FIG. 5B). Gal-1-induced apoptosis thus has ist own profile with involvement of caspases-3, -8 and -9. Assays with Gal-7, another homodimeric proto-type galectin, similarly revealed pro-apoptotic activity of the stimulated T cells. Inhibition of caspases-1, -3 and -8, but not of caspase-9 reduced its extent (see above). Evidently, caspase involvement is different among the proto-type subgroup of galectins, warranting further study. Here, the present inventors focus on the pathway of inducing apoptosis for Gal-2. With caspase-9 as involved mediator of the determined response to Gal-2 exposure, it appeared likely that Gal-2 makes use of the intrinsic pathway, a suggestion testable by looking at the mitochondrial membrane potential. Thus, the inventors next proceeded to analyze this parameter exploiting the redistribution of rhodamine 123.

Galectin-2 induces caspase-3 and -9, but not -8, activity (A). Cells were incubated in the presence or absence of 50 μg/ml galectin-2 for 24 h and caspase activity was assessed by flow cytometry as described in Material and Methods. Illustrated plots are representative for four independent experiments which yielded comparable results. The pan-caspase inhibitor zVAD and the caspase-3 inhibitor (zDEVD) impair the activity of galectins-2, -7 and -1 to induce cell death. The caspase-1 inhibitor zYVAD blocked only gal-7 induced cell death, whereas the caspase-9 inhibitor zLEHD blocked gal-2 and -1 induced apoptosis. The caspase-8 inhibitor zIETD blocked galectin-7 and -1 induced cell death (B). Data represent mean ±SEM of four independent series of experiments. *p<0.05 for increase vs. no presence of galectin; ⁺p<0.05 for decrease vs. galectin.

### Galectin-2 Modifies the Bax/Bcl Ratio and Disrupts the Mitochondrial Membrane Potential

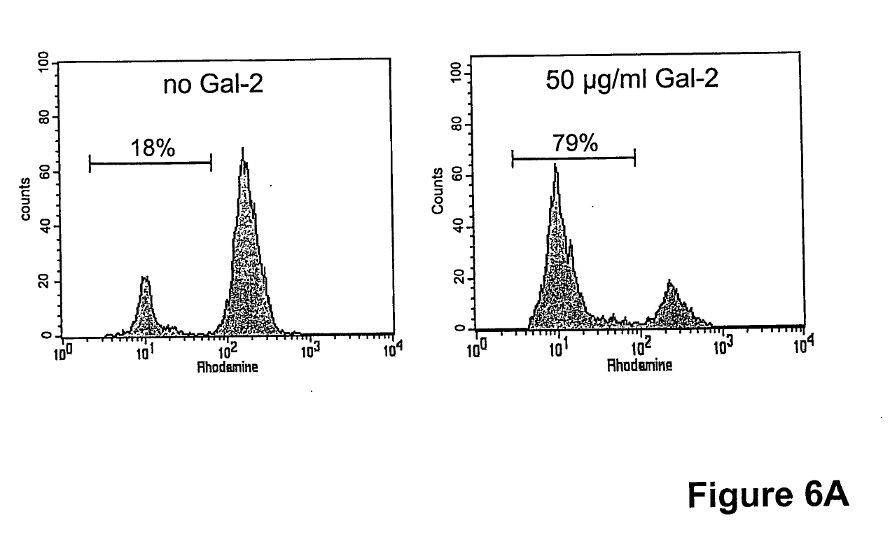

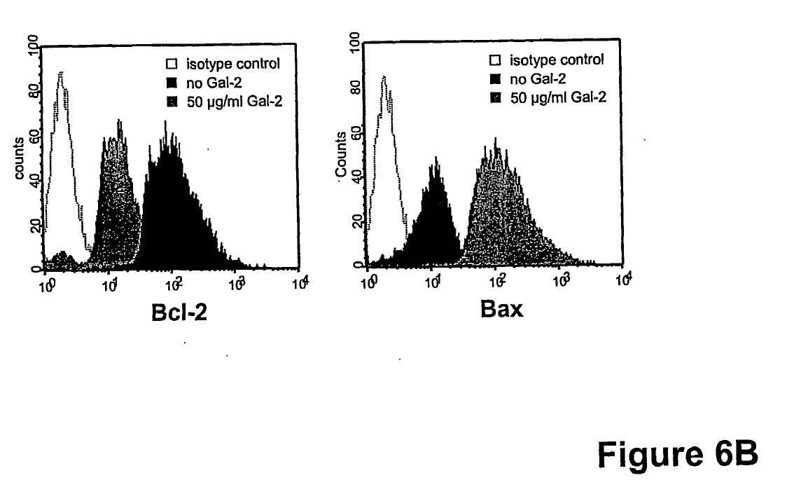

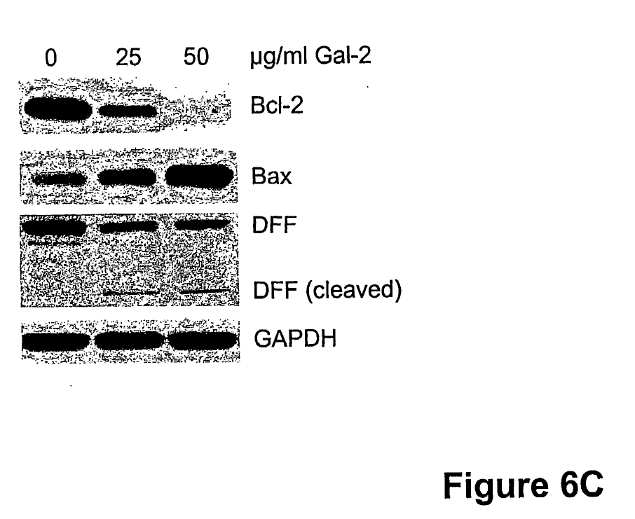

Events critical for apoptosis include the loss of mitochondrial membrane potential, resulting in cytochrome c release and caspase-9 cleavage (29). Having established that galectin-2 induces T cell apoptosis via caspase-9, the inventors next wanted to examine if galectin-2 disrupts the mitochondrial membrane potential, releases cytochrome c and thus uses the “intrinsic”apoptotic pathway. To investigate this possibility, the inventors used the redistribution of the dye rhodamine 123, a lipophilic cation that accumulates in the mitochondrial membrane. When PBT were activated via the CD3 pathway in the presence of 50 μg/ml galectin-2, a clear decrease in the mitochondrial polarization potential compared to cells cultured in the absence of galectin-2 was found (FIG. 6A). Furthermore, galectin-2 dose-dependently increased cytochrome c release in activated, but not resting T cells (data not shown). Apoptosis and survival are also regulated by the relative balance of pro-apoptotic and anti-apoptotic Bcl-2 family members. In addition, since the bax protein forms ion-conducting channels in the lipid bilayers of mitochondria and therefore plays a crucial role in the mitochondrial pathway of apoptosis (30), the inventors investigated if galectin-2 modulates protein expression of the anti-apoptotic bcl-2 and the pro-apoptotic bax proteins in T cells. Immunoblotting and flow cytometry revealed that galectin-2 significantly reduces bcl-2, but increases bax protein expression, resulting in a lower bcl-2/bax ratio (FIGS. 6B and C). It has recently been published that galectin-1 induces surface exposure of phosphatidylserine without inducing apoptosis (31), however other groups could not confirm this finding in other cell types (32, 33). To clarify this discrepancy with respect to galectin-2 and PBT, the inventors analyzed downstream pathways of caspase-3 cleavage in T cells. Immunoblotting revealed, that galectin-2 dose-dependently upregulated the DNA-fragmentation factor (DFF) (FIG. 6C), which is proteolytically cleaved by caspase-3 and irreversibly induces DNA fragmentation and thus cell death (34). These data were confirmed by DAPI staining, showing that 25 and 50 μg galectin-2 induce nuclear fragmentation (data not shown) and confirm the execution of apoptosis by galectin-2.

### Galectin-2 does not Modulate PBT Cell Cycling

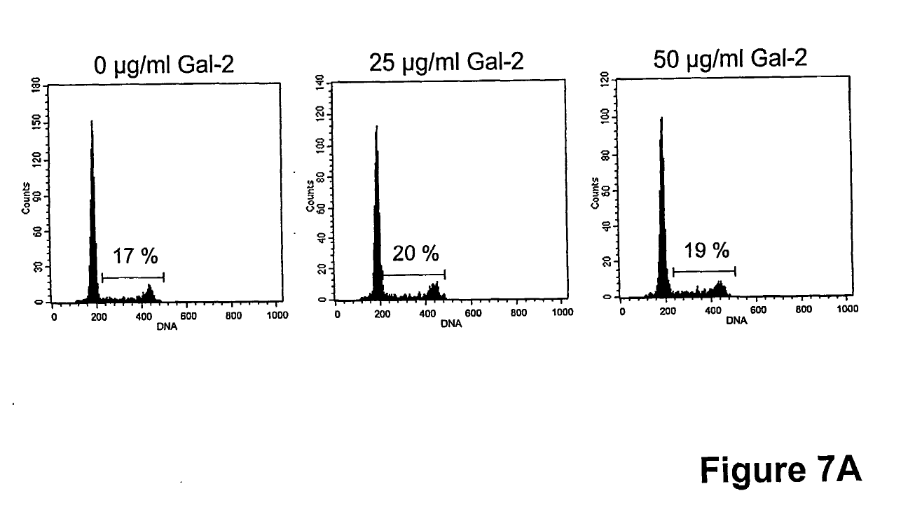

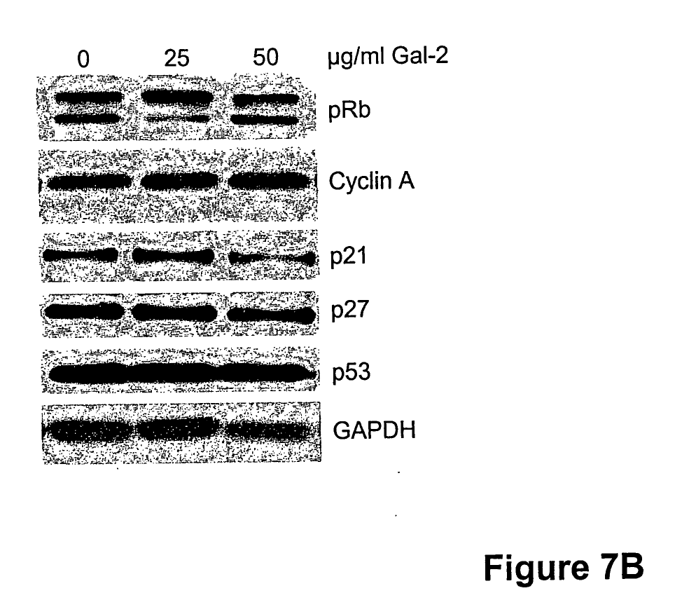

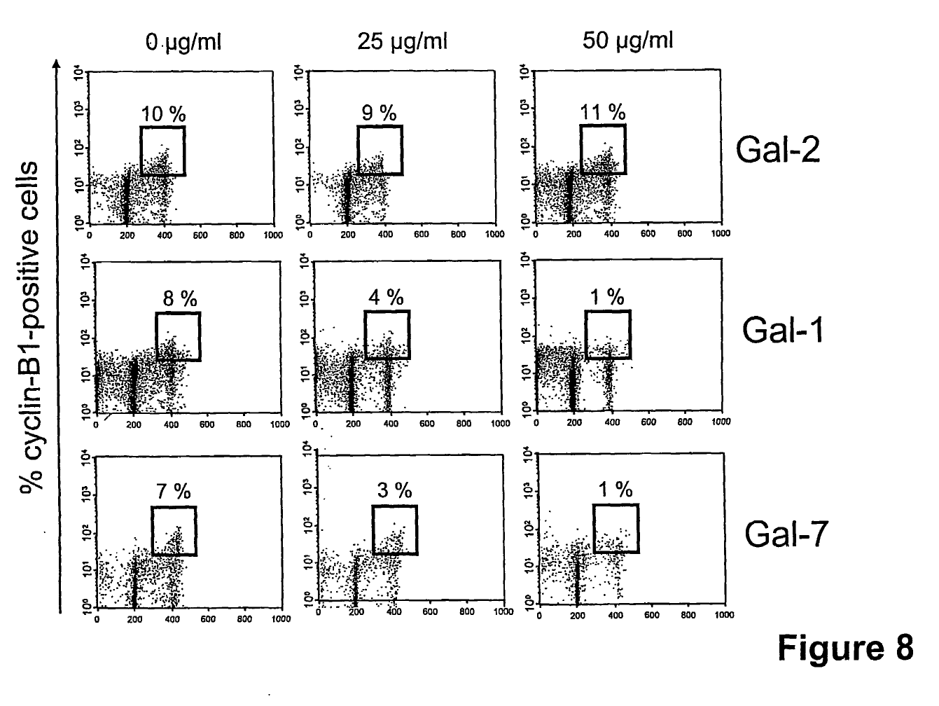

Resting T cells are protected from antigen induced cell death and thus apoptosis and cell cycle are closely linked with each other (35-37). It is known that galectin-1 inhibits proliferation in murine T cell hybridoma and thymocytes (38). To address the question, if galectin-2 modulates T cell cycling, the inventors performed an analysis of PBT cycling profiles in response to galectin-2, using DNA staining. When gated on the living cell fraction, cell cycle phase distribution was comparable in anti-CD3 activated PBT cultured in the absence or presence of 25, 50, and 100 μg/ml galectin-2 (FIG. 7A). The present inventors next investigated if galectin-2 modulates levels of key regulatory molecules responsible for initiating and advancing each phase of the cell cycle. As determined by western blot analysis, galectin-2 did not alter protein levels of the cell cycle promoters cyclin D2, retinoblastoma protein (Rb), or cyclin A, neither the cell cycle inhibitors 21, p27, and p53 (FIG. 7B). To determine if this effect is distinctive for galectin-2, the inventors also examined the effects of galectin-1, -2, and -7 on cyclin B1 expression by flow cytometry in conjunction with PI staining to accurately quantify cell cycle progression to the G2/M-phase. When PBT were activated with anti-CD3 mAb and incubated in the presence of galectin-1 or -7, cyclin B1 expression significantly dropped dose-dependently from 8% to 1% and from 7% to 1%, respectively (FIG. 8). In contrast, galectin-2 did not inhibit PBT cell cycle progression, as demonstrated by unchanged cyclin B1 levels (FIG. 8).

### Gal-2 Distinctively Modulates Cytokine Secretion

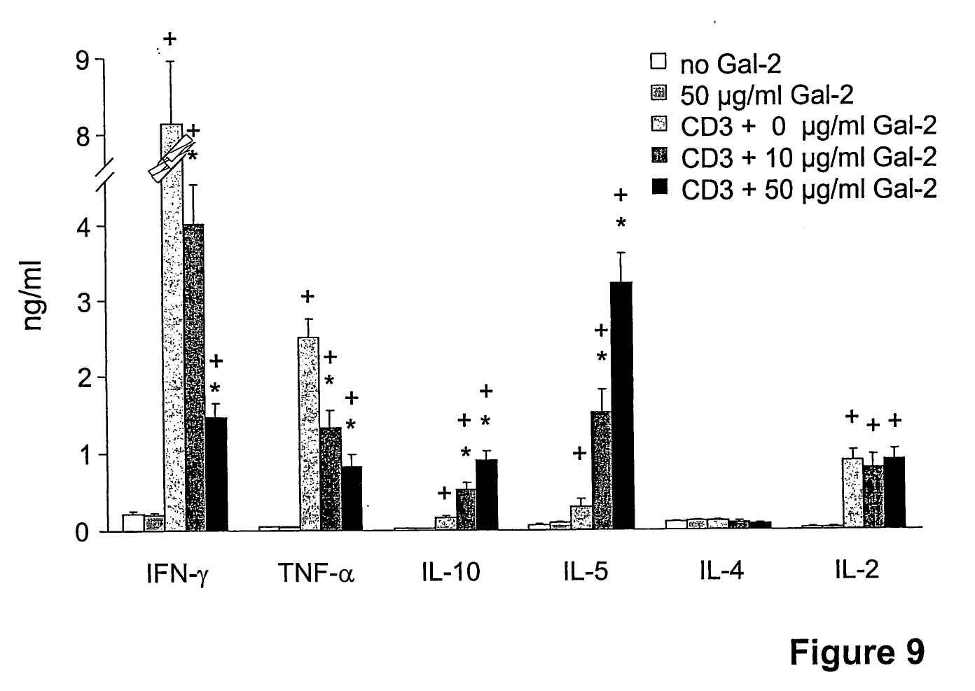

Towards this end the present inventors performed a cytometric bead array using cytokine capture beads followed by flow cytometry. Gal-2 failed to modulate cytokine production of resting T cells. Activation of T-cells with anti-CD3 mAb profoundly upregulated IFN-γ, TNF-α, IL-10, IL-5 and IL-2 production (FIG. 9). In activated T-cells, Gal-2 downregulated significantly and dose-dependently IFN-γ and TNF-α production, while increasing IL-5 and IL-10 secretion (FIG. 9). IL-4 secretion did not change upon T-cell activation and was not altered by presence of Gal-2. In contrast, IL-2 secretion was upregulated by cell activation but, as could be expected in view of the lack of a Gal-2-dependent effect on T cell cycling, it was not influenced by presence of Gal-2 (FIG. 9). Having hereby examined cell cycle and cytokine parameters, the inventors finally turned to analyze Gal-2-dependent effects on the treated cells' capacity to recruit integrins as mediators of adhesion and outside-in signaling.

### Galectin-2 Modulates Integrin Accessibility and Cell Adhesion

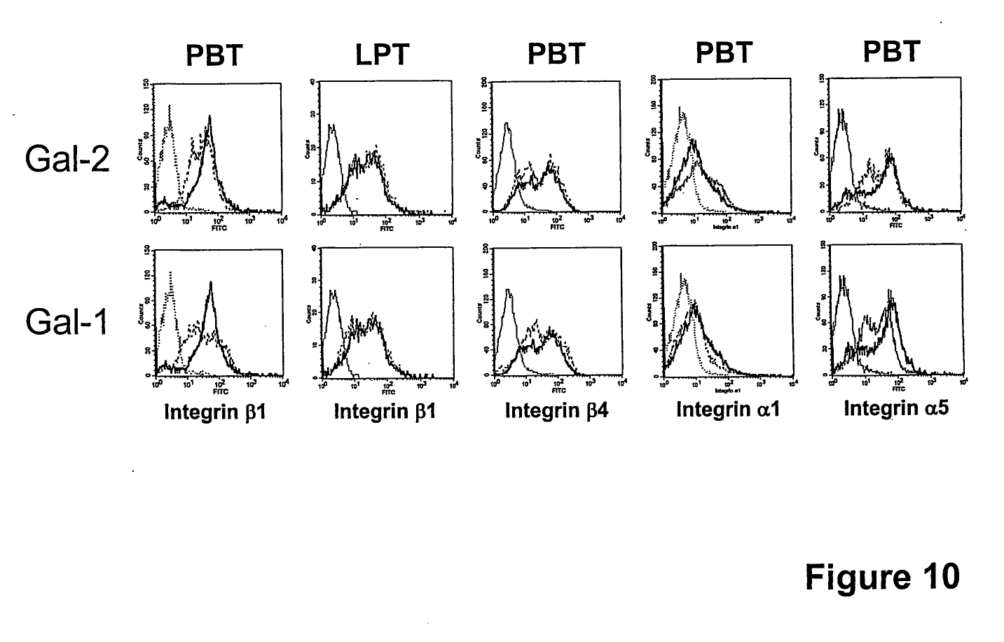

Galectins act as modulators of cell adhesion by interacting with the appropriately glycosylated proteins at the cell surface or within the extracellular matrix (ECM) (39). This interaction is mediated by integrins and since the inventors showed above, that galectin-2 bound to integrin 1, the inventors wanted to determine, if galectin-2 alters integrin accessibility on T cells. Treatment of the activated T cells had a bearing on integrin reactivity to antibodies, a measure of accessibility. When PBT are activated via the CD3 pathway, galectin-1 and -2 downregulated integrin β1 and α5 accessibility (FIG. 10). In contrast, neither galectin-1 nor galectin-2 changed β4 integrin accessibility, responsible for T cell homing to the intestinal lamina propria, and slightly upregulated integrin a5 accessibility, indicating that galectins regulate integrin accessibility distinctively. Interestingly, when intestinal T cells as source of memory T cells were examined, galectin-2 failed to modulate integrin β1 accessibility, indicating that the ability of galectin-2 to affect integrin accessibility is distinct not only for individual integrins, but also for cell differentiation (FIG. 10).

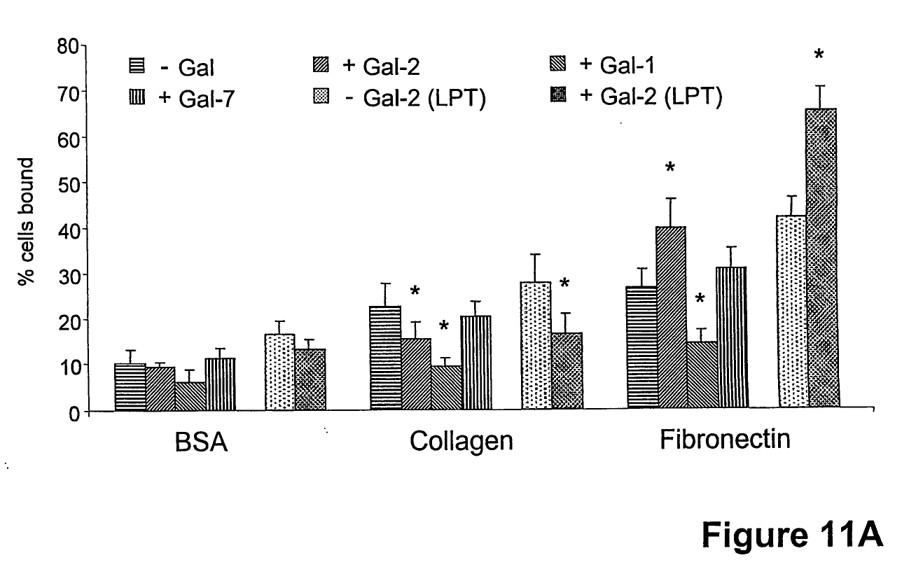

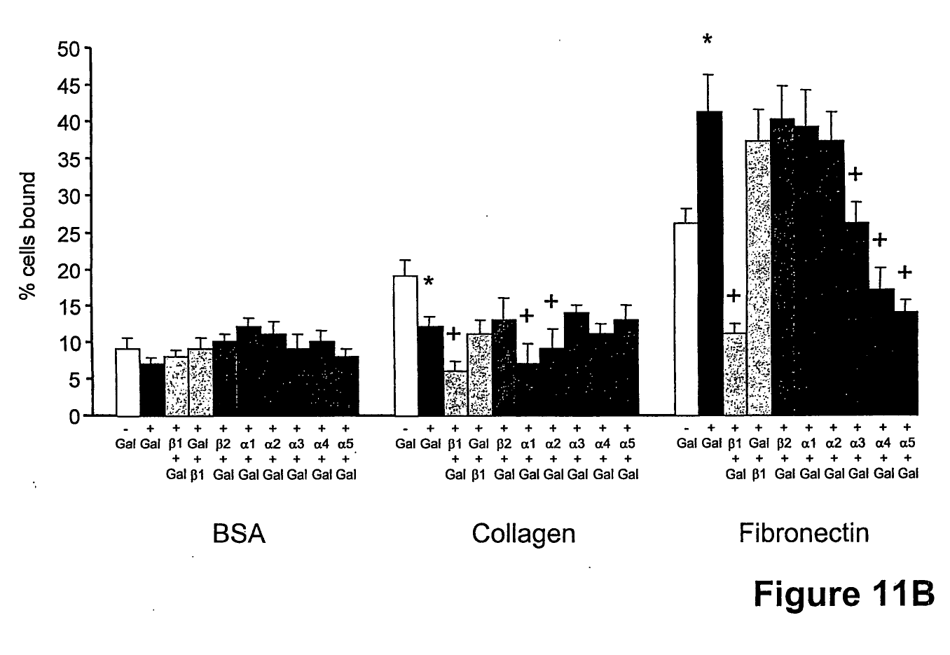

Galectins distinctively modulate T cell adhesion to extracellular matrix and the presence of galectin-1 inhibits T cell adhesion to ECM glycoproteins (40, 41). Having shown that galectin-2 alters integrin accessibility of T cells, the inventors hypothesized that galectin-2 will also modify T cell adherence to ECM compounds. When PBT were activated via the CD3 pathway, 22.1±3.5% and 26.3±5.6% of the cells adhere to collagen type I or fibronectin, respectively (FIG. 11Aa). Confirming the known effect of galectin-1 on T cell adhesion (40), galectin-1 substantially decreased T cell attachment to collagen type I (9.2±3.5%) and fibronectin (14.0±4.6%) (FIG. 11A). In contrast, while adhesion to collagen type I was also significantly reduced by galectin-2 (15.1±5.6%; p<0.05), adhesion to fibronectin was significantly enhanced by galectin-2 (39.8±4.9%; p<0.05) (FIG. 11A). Again, the inventors analysed galectin-7 to verify if this feature is specific for galectin-2. As depicted in FIG. 11A, galectin-7 failed to significantly alter PBT adhesion to collagen or fibronectin. As shown above, integrin β1 upregulation by CD3 is inhibited by galectin-2 in PBT, but not LPT. However, when the effect of galectin-2 on T cell adhesion was analysed in LPT, comparable to PBT, galectin-2 inhibited cell adhesion to collagen, while increasing attachment to fibronectin (p<0.05; FIG. 11A). To confirm that these effects were mediated by collagen and fibronectin receptors, cells were pre-incubated for 1 h with a subset of specific blocking integrin mAbs. After pre-incubation, 25 μg/ml galectin-2 was added and cells were layered on BSA-, collagen- or fibronectin-coated wells. Cell adhesion to collagen was significantly inhibited by β1, α1 and α2 mAbs, whereas the adhesion to fibronectin was blocked by β1, α3, α4, and α5 mAbs (FIG. 11B), indicating that the adhesion of cells to their respective extracellular matrix was mediated by collagen and fibronectin receptors, respectively. As shown above, galectin-2 binds to β1 (FIGS. 3A, B). To determine, if this binding may involve β1 integrin on the cell surface and may therefore alter PBT adhesion to ECM, the inventors pre-incubated cells for 1 h with galectin-2 and added integrin-β1 mAbs. Cells were then allowed to adhere on the ECM compounds. Interestingly, pre-incubation with galectin-2 before adding integrin-β1 mAb reversed the inhibiting effect of the integrin β1 mAb, when they were added first, indicating that, at least in part, integrin accessibility sites were neutralized by galectin-2 (FIG. 11B).

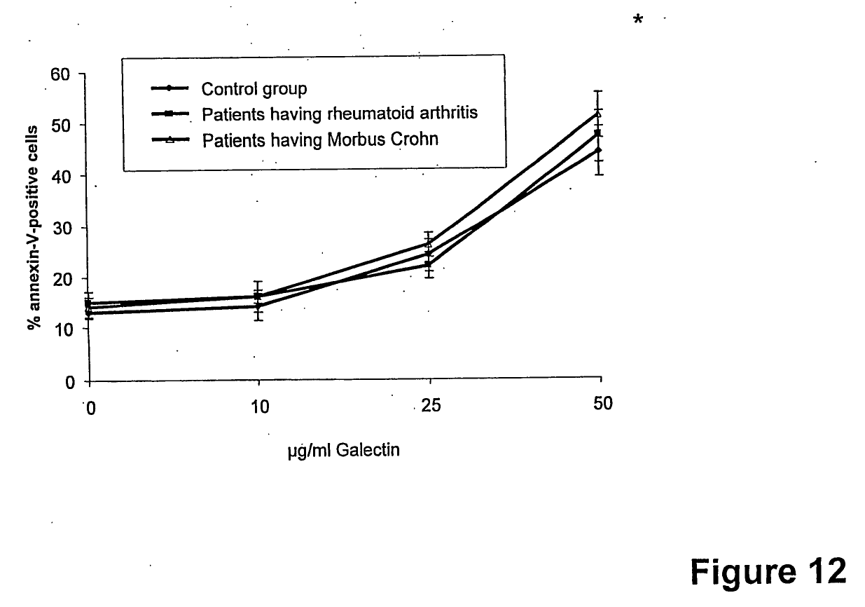

Galectin-2 induces apoptosis in T-cells from patients having rheumatoid arthritis (RA) or Crohn's disease (CD). PBT from patients with CD and RA and from healthy control group were stimulated with cross-linked anti-CD3 mAb antibodies for 48 hours in the presence of 0, 10, 25 or 50 μg/ml human galectin-2. Apoptosis was determined by annexin-V-staining (FIG. 12).

### Example 3

### Prevention of Colitis

### Prevention with 1 Mg/Kg Body Weight

Colitis was induced in 8- to 10-week-old, female, Balb/c mice (Jackson Laboratories, Bar Harbor, Me.) by adding 5% DSS (Sigma Chemical Co., St. Louis, Mo.) (Dextran Sodiumsulfate (DSS) to their drinking water and allowing them to drink ad libitum. Groups of mice were treated either with 1 mg/kg BW (body weight) galectin-2 intraperitoneally or PBS 2 h before DSS administration. Mice were weighed and inspected for diarrhea and rectal bleeding. Seven days after induction of colitis by DSS, mice were killed, their entire colon was resected, and its length was measured and weighed. The disease activity index (DAI; i.e., the combined score of weight loss and bleeding) was determined according to a standard scoring system.

The disease activity index:
Loss in bodyweight: 0=no loss; 1=5% to 10%; 2=10% to 15%; 3=15% to 20%; 4 [symbol] 20%.

### Hemoccult: 0=no blood; 2=positive; 4=gross blood.

In this experiment the DAI ranges from 0 to 8. The higher the DAI score the more severely the animal had been affected by colitis.

### Results:

| DAI: Control group: | 6.3 ± 2.30 (mean ± SD), | n = 24 | |
|---|---|---|---|
| Galectin group: | 4.5 ± 2.1, | n = 10; | p = 0.04 |

The results show that those animals treated with galectin-2 showed a significant reduction of severity of colitis proving that galectin-2 is particularly suitable for the prevention or treatment of inflammatory bowel diseases.

### Prevention with 2 mg/kg BW

Colitis was induced in 8- to 10-week-old, female, Balb/c mice (Jackson Laboratories, Bar Harbor, Me.) by adding 5% DSS (Sigma Chemical Co., St. Louis, Mo.) (Dextran Sodiumsulfate (DSS) to their drinking water and allowing them to drink ad libitum. Groups of respectively 10 mice were treated either with Galectin-2 subcutaneously (s.c.) or intraperitoneally (i.p.) 2 h before DSS administration. In one trial with 10 mice the doses of Galectin-2 was doubled (administered i.p.) and compared to the treatment of 10 mice with Tacrolimus (FK506) (administered i.p.) in equal concentrations. In parallel a group of 10 mice was treated with 0.9% NaCl instead of Galectin-2 as control. Mice were weighed and inspected for diarrhea and rectal bleeding. 10 days after induction of colitis by DSS, mice were killed, their entire colon was resected, and its length was measured and weighed. The disease activity index (DAI; i.e., the combined score of weight loss and bleeding) was determined according to a standard scoring system.

The disease activity index:
Loss in body weight: 0=no loss; 1=5% to 10%; 2=10% to 15%; 3=15% to 20%; 4 [symbol] 20%.

### Hemoccult: 0=no blood; 2=positive; 4=gross blood.

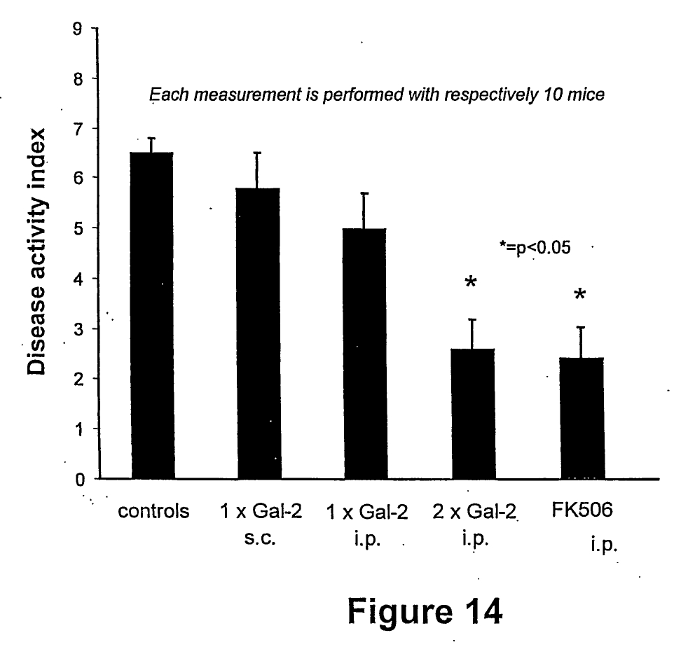

### Results (also shown in FIG. 14):

| | | | |
|---|---|---|---|
| DAI: Control group: | 6.5 ± 0.3 | n = 10 | |
| | (mean ± SD), | | |
| Galectin group 1 mg/kg s.c.: | 5.8 ± 0.7, | n = 10; | p < 0.05 |
| Galectin group 1 mg/kg i.p.: | 5.0 ± 0.7, | n = 10; | p < 0.05 |
| Galectin group 2 mg/kg i.p.: | 2.6 ± 0.6, | n = 10; | p < 0.05 |
| Tacrolimus group 2 mg/kg i.p.: | 2.4 ± 0.5, | n = 10; | p < 0.05 |

The results show that those animals treated with galectin-2 intraperitoneally showed better results than those treated subcutaneously. Animals treated with 2 mg/kg showed significant reduction of severity of colitis with results in the same range like results from treatment with FK 506, proving that galectin-2 is particularly suitable for the prevention or treatment of inflammatory bowel diseases.

### Example 4

### Therapy of Chronic Colitis

### Therapy with 2 mg/kg BW

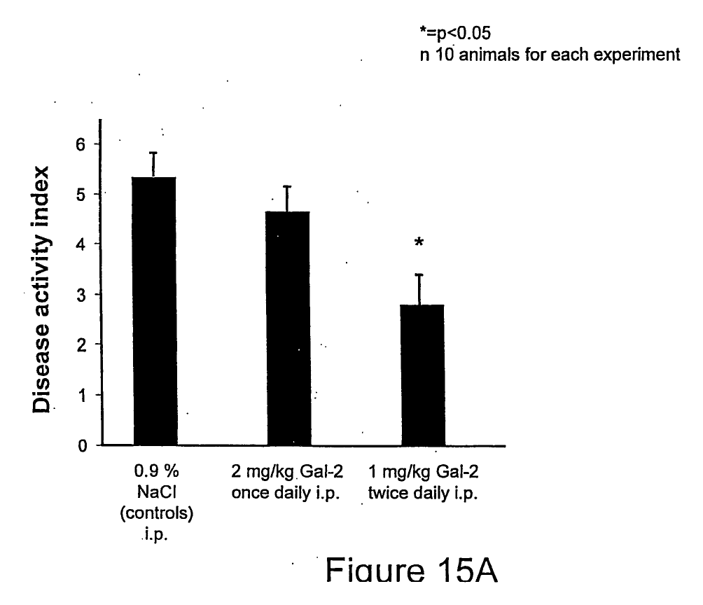

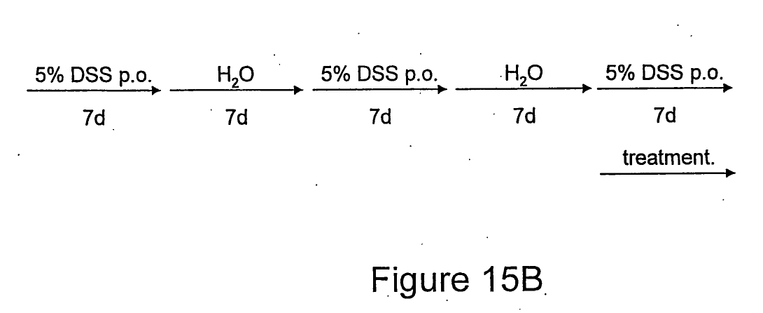

Colitis was induced in 30 8- to 10-week-old, female, Balb/c mice (Jackson Laboratories, Bar Harbor, Me.) by adding 5% DSS (Sigma Chemical Co., St. Louis, Mo.) (Dextran Sodiumsulfate (DSS) to their drinking water for the period of seven days and allowing them to drink ad libitum. At the 8th day DSS administration was stopped for another seven days. This scheme of DSS administration for seven days followed by a period of seven days pure water administration was repeated as shown in FIG. 15B. At the 29th day 20 mice were treated with Galectin-2 intraperitoneally (i.p.) parallel to DSS administration. In one trial of this treatment a group of 10 mice were treated with 2 mg/kg Galectin-2 once a day and in another trial the same total dose was distributed in two administrations for a group of ten mice, e.g. 10 mice were treated with respectively 1 mg/kg Galectin-2 twice a day. Parallel a group of 10 mice was treated with 0,9% NaCl instead of Galectin-2 as control. At the 8th day the Galectin-2 treatment was stopped. Parallel to the treatment mice were weighed and inspected for diarrhea and rectal bleeding. After stopping the treatment mice were killed, their entire colon was resected, and its length was measured and weighed. The disease activity index (DAI; i.e., the combined score of weight loss and bleeding) was determined according to a standard scoring system

The disease activity index:
Loss in bodyweight: 0=no loss; 1=5% to 10%; 2=10% to 15%; 3=15% to 20%; 4 [symbol] 20%.

### Hemoccult: 0=no blood; 2=positive; 4=gross blood.

### Results (also shown in FIG. 15A:

| | | | |
|---|---|---|---|
| DAI: Control group: | 5.3 ± 0.7 | n = 10 | |
| | (mean ± SD), | | |
| Galectin group 1 × 2 mg/kg: | 4.5 ± 0.6, | n = 10; | p < 0.05 |
| Galectin group 2 × 1 mg/kg: | 2.8 ± 0.3, | n = 10; | p < 0.05 |

The results show that those animals treated with galectin-2 intraperitoneally twice a day showed significantly better results than those treated with the same total dose once a day. Animals treated with 1 mg/kg twice a day showed significant reduction of severity of colitis, proving that galectin-2 is particularly suitable for the therapy of inflammatory bowel diseases.

### Example 5

### Dose Finding Studies

Acute and chronic colitis were induced in BALB/c mice by adding 5% DSS to the drinking water for 7 days to induce acute colitis, or for 3 cycles of 7 days DSS following 7 days without DSS to induce chronic colitis, respectively (Siegmund B, Lehr H A, Fantuzzi G. Leptin: a pivotal mediator of intestinal inflammation in mice. Gastroenterology 2002; 122: 2011-2025). Animals with acute or chronic colitis were treated i.p. with either physiological NaCl as control or Gal-2 once daily (od), twice daily (bid) or three time daily (tid) for 10 days. In one experimental protocol Gal-2 and NaCl 0.9% were injected subcutaneously. Gal-2 doses ranging from 0.5-2 mg/kg body weight in individual doses and total daily doses of 3 mg/kg body weight and different dosing regimens were evaluated. Tacrolimus (1 mg/kg bodyweight), Infliximab (10 mg/kg bodyweight), prednisolone (1 mg/kg bodyweight), all well-known modulators of experimental colitis and human inflammatory bowel diseases were used as positive controls.

PBT were isolated, stimulated with CD3 for 3 days and cytokine secretion was determined by cytometric bead array (CBA) analysis as described previously (Sturm A, Rilling K, Baumgart D C, Gargas K, Abou-Ghazale T, Raupach B, Eckert J, Schumann R R, Enders C, Sonnenborn U, Wiedenmann B, Dignass A U. Escherichia coli Nissle 1917 distinctively modulates T-cell cycling and expansion via toll-like receptor 2 signaling. Infect.Immun. 2005; 73: 1452-1465.) Mucosal recruitment of granulocytes was assessed by Myeloperoxidase activity (Rath H C, Herfarth H H, Ikeda J S, Grenther W B, Hamm T E, Jr., Balish E, Taurog J D, Hammer R E, Wilson K H, Sartor R B. Normal Luminal Bacteria, Especially Bacteroides Species, Mediate Chronic Colitis, Gastritis, and Arthritis in HLA-B27/Human beta 2 Microglobulin Transgenic Rats. Journal of Clinical Investigation 1996; 98: 945-953).

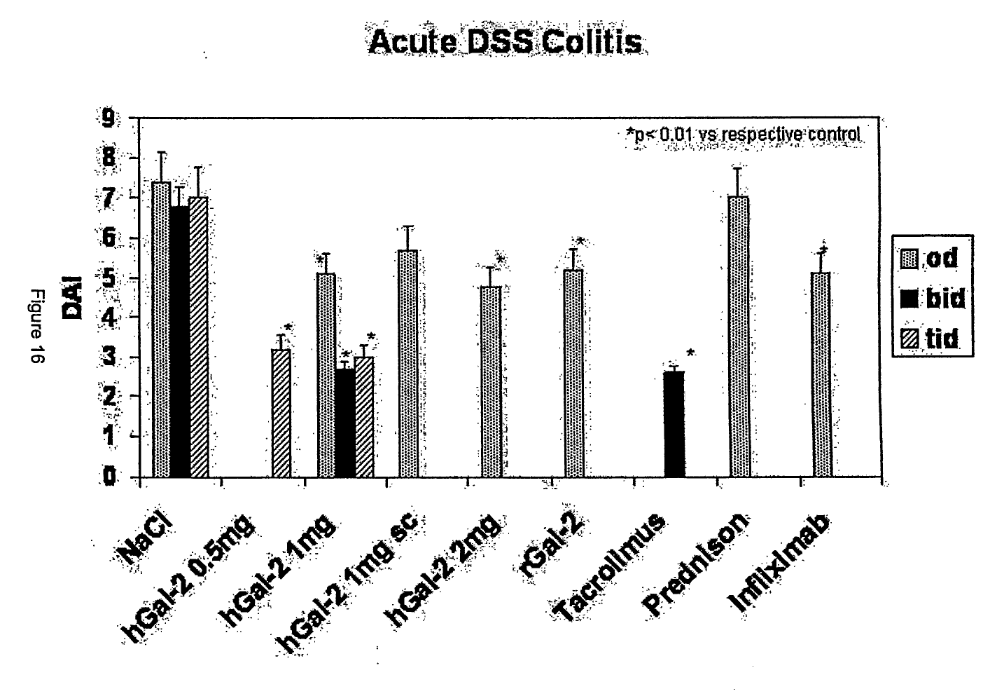

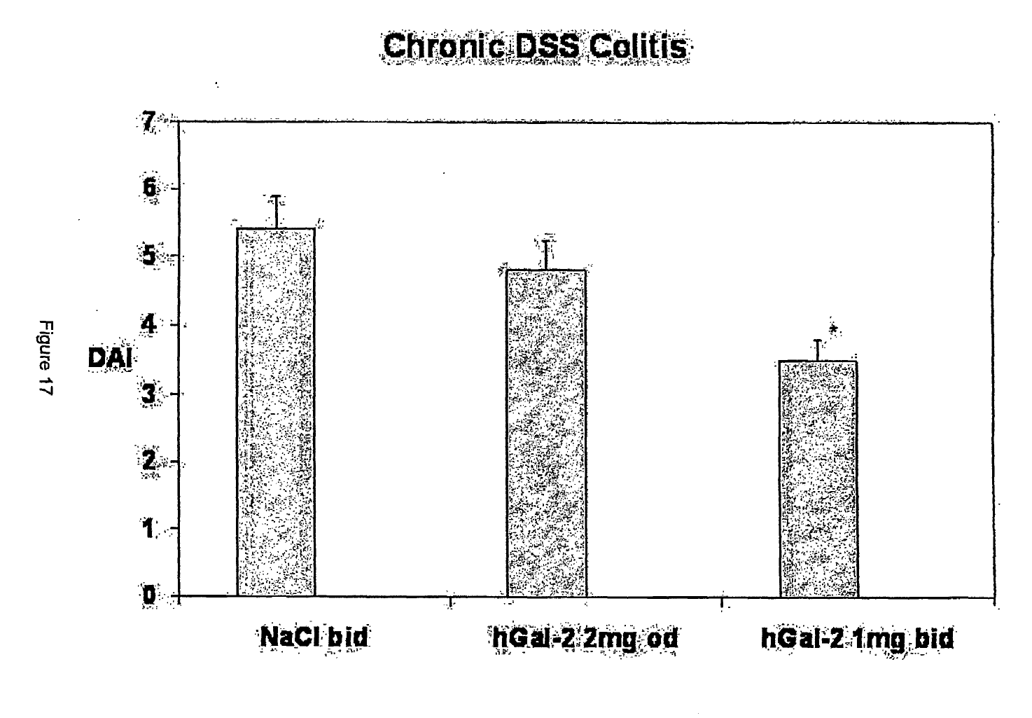

Treatment of mice with acute and chronic DSS colitis with Gal-2 significantly reduced disease activity (as measured by disease activity index (DAI), as outlined above) in both acute and chronic DSS induced colitis in mice using different dosing intervals and doses (FIGS. 16 and 17). An optimal response was observed with bid dosing and could not be further improved with tid dosing. Subcutaneous (sc) administration of Gal-2 resulted only in minor and not significant improvement of disease activity (FIG. 16). Looking at total daily doses, the dose range from 1.5-3 mg/kg bodyweight Galectin-2 per day provided the largest benefit with respect to improvement of disease activity, while the greatest effect was observed for a total dose of 2 mg/kg bodyweight daily administered in two daily doses of 1 mg/kg bodyweight. No significant differences were observed, when equivalent doses and treatment regimens of human and rat Gal-2 were compared. The effects of Gal-2 were comparable with the potent immunomodulator tacrolimus and more potent as the effects observed for prednisolone and infliximab. With the optimal dose of 2×1 mg/kg bodyweight disease activity was improved on average more than 250%. Histological analyses demonstrated a significant reduction of intestinal injury and inflammation in Gal-2 treated mice compared to control animals (FIG. 18). This was further corroborated by myeloperoxidase measurements revealing a significant reduction of mucosal granulocyte infiltration in Gal-2 treated mice (data not shown).

Safety and toxicity of Galectin-2 was also assessed in the above described dose finding studies in the acute and chronic DSS model of colitis and did not reveal any abnormalities. In addition, in all of these studies, no deaths were observed in mice with DSS induced acute or chronic colitis that were treated with Galectin-2 doses ranging from 1-3 mg/kg body weight.

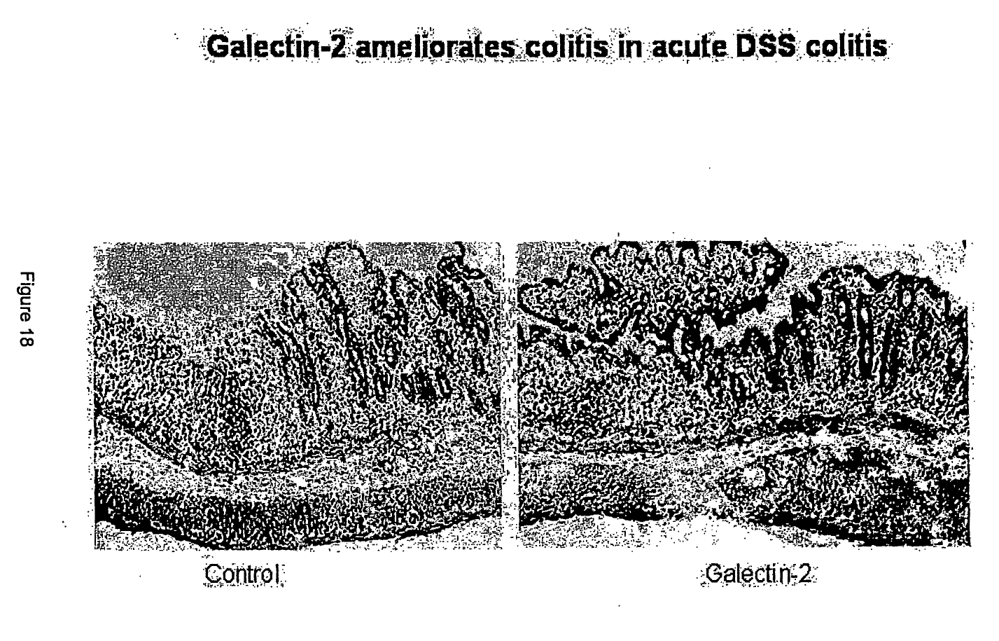

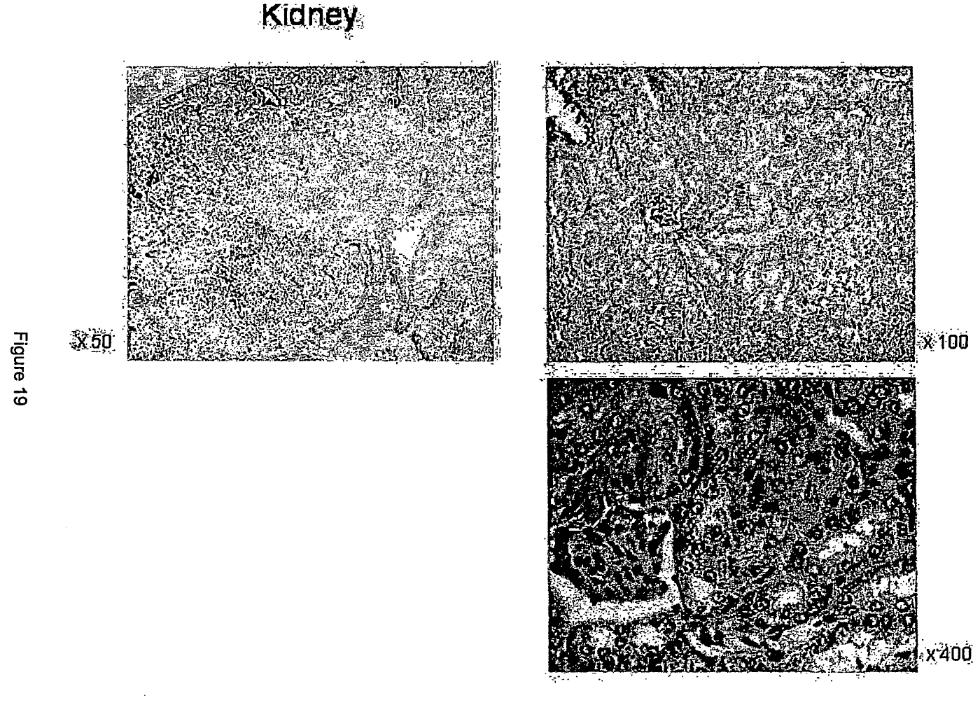

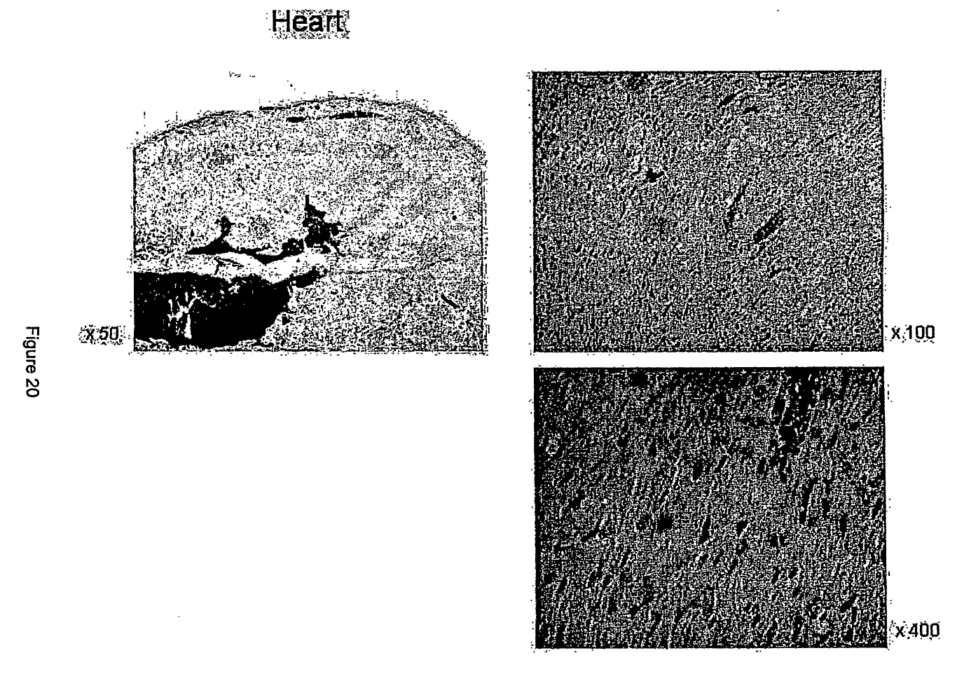

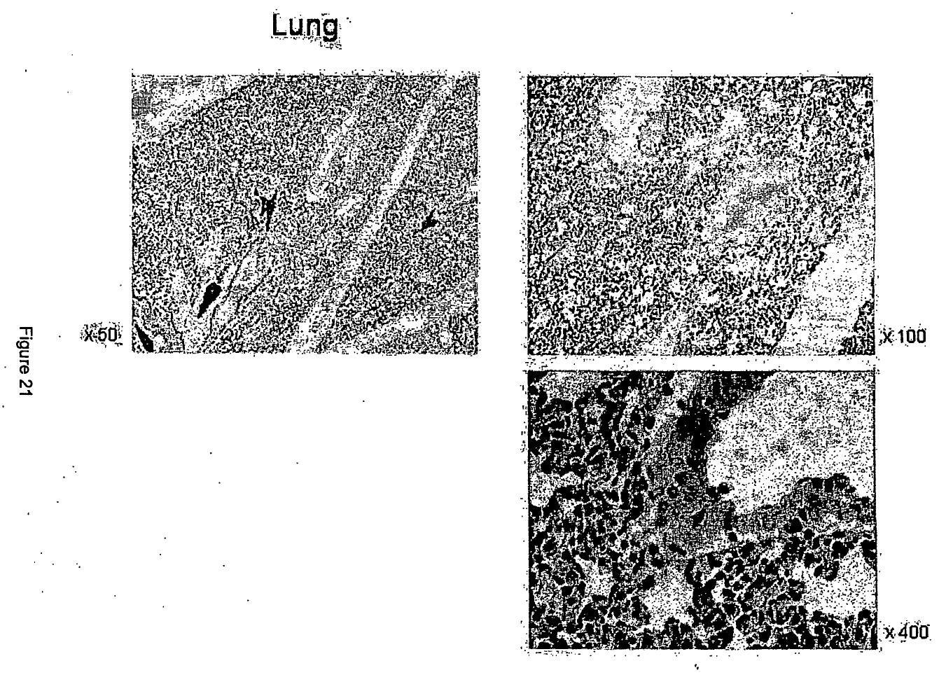

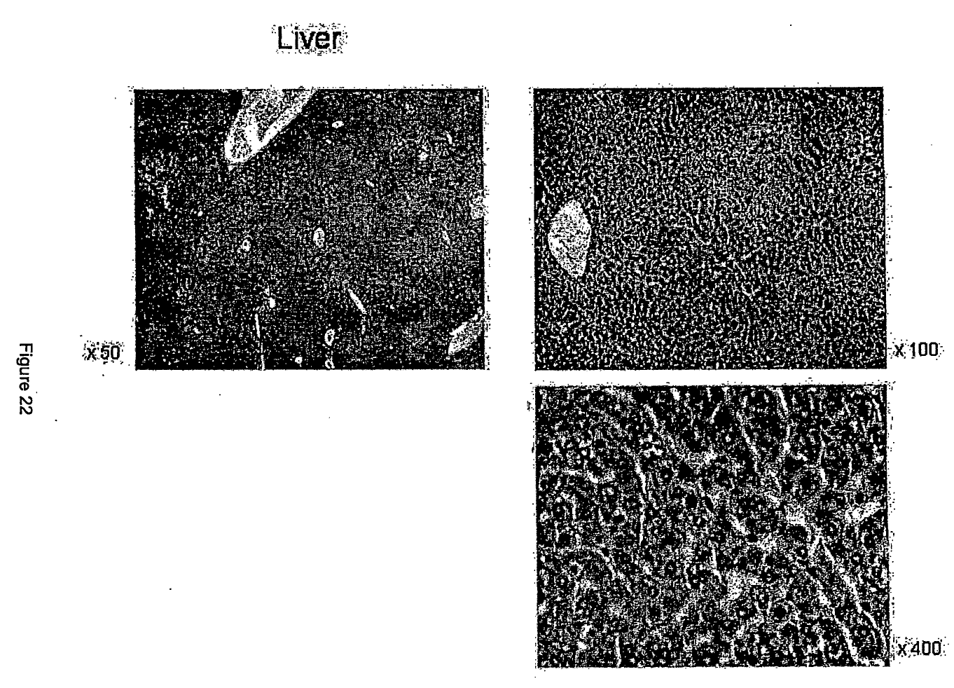

In addition, Balb/c mice (not treated with DSS) were treated with 100 mg/kg body weight-Galectin-2, a dose that was up to 50-fold higher than doses of Galectin-2 that caused optimal anti-inflammatory effects in DSS treated mice (optimal therapeutic doses ranged from 1.5-3 mg/kg body weight per day). In these toxicity studies Balb/c mice (n=5), weighing on average 20 g, were treated with 100 mg/kg body weight Galectin-2 once daily i.p. for 5 days. The mice gained about 7±1.7% weight during the 5 day treatment period. None of these animals died. The mice were sacrificed following the 5 day treatment period and organs were collected for pathological assessment by an experienced pathologist. Pathological assessment did not reveal any microscopic or macroscopic damage in the heart, kidney, lung, or liver as depicted in FIGS. 19-22.

In summary, no dose limiting toxicities were observed for galectin-2 over a broad dose range. The highest dose of 100 mg/kg body weight galectin-2 evaluated is about 50-fold higher than the optimal dose of 2×1 mg/kg bodyweight galectin-2 for the treatment of DSS induced colitis.

Therefore, a therapeutic role of Gal-2 was substantiated by its potent anti-inflammatory and regenerative effects in an experimental mode of murine colitis (DSS colitis), while no apparent toxicity was observed for therapeutic and even supra-therapeutic doses of Gal-2. These findings suggest that Gal-2 provides a new approach in the treatment of diseases with an impaired T cell apoptosis, e.g. inflammatory bowel diseases.

The features of the present invention disclosed in the specification, the claims and/or in the accompanying drawings, may, both separately, and in any combination thereof, be material for realising the invention in various forms thereof.

### REFERENCES

1. Gabius, H.-J., S. André, H. Kaltner, and H. C. Siebert. 2002. The sugar code: functional lectinomics. Biochim.Biophys.Acta 1572:165.
2. Rabinovich, G. A., L. G. Baum, N. Tinari, R. Paganelli, C. Natoli, F. T. Liu, and S. Iacobelli. 2002. Galectins and their ligands: amplifiers, silencers or tuners of the inflammatory response? Trends Immunol. 23:313.
3. Cooper, D. N. and S. H. Barondes. 1999. God must love galectins; he made so many of them. Glycobiology 9:979.
4. Liu, F. T. 2000. Galectins: a new family of regulators of inflammation. Clin.Immunol. 97:79.
5. Rabinovich, G. A. 1999. Galectins: an evolutionarily conserved family of animal lectins with multifunctional properties; a trip from the gene to clinical therapy. Cell Death.Differ. 6:711.
6. Rabinovich, G. A., N. Rubinstein, and M. A. Toscano. 2002. Role of galectins in inflammatory and immunomodulatory processes. Biochim.Biophys.Acta 1572:274.
7. Gitt, M. A., E. K. Jordan, and H. Leffler. 1997. Galectin-2, Galectins-5 and -9, and Galectins-4 and -6. Trends Glycosci.Glycotechnol. 9:87.
8. Lahm, H., S. André, A. Hoeflich, J. R. Fischer, B. Sordat, H. Kaltner, E. Wolf, and H. -J. Gabius. 2001. Comprehensive galectin fingerprinting in a panel of 61 human tumor cell lines by RT-PCR and its implications for diagnostic and therapeutic procedures. J. Cancer Res. Clin. Oncol. 127:375.
9. Oka, T., S. Murakami, Y. Arata, J. Hirabayashi, K. Kasai, Y. Wada, and M. Futai. 1999. Identification and cloning of rat galectin-2: expression is predominantly in epithelial cells of the stomach. Arch.Biochem.Biophys. 361:195.
10. Marschal, P., V. Cannon, S. H. Barondes, and D. N. Cooper. 1994. Xenopus laevis L-14 lectin is expressed in a typical pattern in the adult, but is absent from embryonic tissues. Glycobiology 4:297.
11. Balomenos, D. and A. Martinez. 2000. Cell-cycle regulation in immunity, tolerance and autoimmunity. Immunol. Today 21:551.
12. Lenardo, M., K. M. Chan, F. Hornung, H. McFarland, R. Siegel, J. Wang, and L. Zheng. 1999. Mature T lymphocyte apoptosis—immune regulation in a dynamic and unpredictable antigenic environment. Annu.Rev.Immunol. 17:221-53.:221.
13. Sherr, C. J. and J. M. Roberts. 1999. CDK inhibitors: positive and negative regulators of G1-phase progression. Genes Dev. 13:1501.
14. Kaltner, H. and B. Stierstorfer. 1998. Animal lectins as cell adhesion molecules. Acta Anat (Basel) 161:162.
15. Boudreau, N. J. and P. L. Jones. 1999. Extracellular matrix and integrin signalling: the shape of things to come. Biochem.J. 339:481.
16. Hemler, M. E. 1990. VLA proteins in the integrin family: structures, functions, and their role on leukocytes. Annu.Rev.Immunol. 8:365-400.:365.
17. Shimizu, Y., G. A. van Seventer, K. J. Horgan, and S. Shaw. 1990. Regulated expression and binding of three VLA (beta 1) integrin receptors on T cells. Nature 345:250.
18. Moiseeva, E. P., B. Williams, A. H. Goodall, and N. J. Samani. 2003. Galectin-1 interacts with beta-1 subunit of integrin. Biochem.Biophys.Res.Commun. 310:1010.
19. Brandtzaeg, P., I. N. Farstad, and G. Haraldsen. 1999. Regional specialization in the mucosal immune system: primed cells do not always home along the same track. Immunol.Today 20:267.
20. Furtak, V., F. Hatcher, and J. Ochieng. 2001. Galectin-3 mediates the endocytosis of beta-1 integrins by breast carcinoma cells. Biochem.Biophys.Res.Commun. 289:845.
21. Sturm, A., J. Itoh, J. W. Jacobberger, and C. Fiocchi. 2002. p 53 negatively regulates intestinal immunity by delaying mucosal T cell cycling. J.Clin.Invest 109:1481.
22. Toescu, E. C. and A. Verkhratsky. 2000. Assessment of mitochondrial polarization status in living cells based on analysis of the spatial heterogeneity of rhodamine 123 fluorescence staining. Pflugers Arch. 440:941.
23. Scaduto, R. C., Jr. and L. W. Grotyohann. 1999. Measurement of mitochondrial membrane potential using fluorescent rhodamine derivatives. Biophys.J. 76:469.
24. Pace, K. E., C. Lee, P. L. Stewart, and L. G. Baum. 1999. Restricted receptor segregation into membrane microdomains occurs on human T cells during apoptosis induced by galectin-1. J.Immunol. 163:3801.
25. Perillo, N. L., K. E. Pace, J. J. Seilhamer, and L. G. Baum. 1995. Apoptosis of T cells mediated by galectin-1. Nature 378:736.
26. Yang, R. Y., D. K. Hsu, and F. T. Liu. 1996. Expression of galectin-3 modulates T-cell growth and apoptosis. Proc.Natl.Acad.Sci.U.S.A 93:6737.
27. Santucci, L., S. Fiorucci, N. Rubinstein, A. Mencarelli, B. Palazzetti, B. Federici, G. A. Rabinovich, and A. Morelli. 2003. Galectin-1 suppresses experimental colitis in mice. Gastroenterology 124:1381.
28. Kashio, Y., K. Nakamura, M. J. Abedin, M. Seki, N. Nishi, N. Yoshida, T. Nakamura, and M. Hirashima. 2003. Galectin-9 induces apoptosis through the calcium-calpain-caspase-1 pathway. J.Immunol. 170:3631.
29. Hengartner, M. O. 2000. The biochemistry of apoptosis. Nature 407:770.
30. Kroemer, G. 2003. Mitochondrial control of apoptosis: an introduction. Biochem.Biophys.Res.Commun. 304:433.
31. Dias-Baruffi, M., H. Zhu, M. Cho, S. Karmakar, R. P. McEver, and R. D. Cummings. 2003. Dimeric galectin-1 induces surface exposure of phosphatidylserine and phagocytic recognition of leukocytes without inducing apoptosis. J. Biol. Chem. 278:41282.
32. Rabinovich, G. A., G. Daly, H. Dreja, H. Tailor, C. M. Riera, J. Hirabayashi, and Y. Chernajovsky. 1999. Recombinant galectin-1 and its genetic delivery suppress collagen-induced arthritis via T cell apoptosis. J.Exp.Med. 190:385.
33. Rabinovich, G. A., M. M. Iglesias, N. M. Modesti, L. F. Castagna, C. Wolfenstein-Todel, C. M. Riera, and C. E. Sotomayor. 1998. Activated rat macrophages produce a galectin-1-like protein that induces apoptosis of T cells: biochemical and functional characterization. J.Immunol. 160:4831.
34. Liu, X., H. Zou, C. Slaughter, and X. Wang. 1997. DFF, a heterodimeric protein that functions downstream of caspase-3 to trigger DNA fragmentation during apoptosis. Cell 89:175.
35. Vaux, D. L. and S. J. Korsmeyer. 1999. Cell death in development. Cell 96:245.
36. King, K. L. and J. A. Cidlowski. 1995. Cell cycle and apoptosis: common pathways to life and death. J Cell Biochem. 58:175.
37. Boehme, S. A. and M. J. Lenardo. 1993. Propriocidal apoptosis of mature T lymphocytes occurs at S phase of the cell cycle. Eur.J.Immunol. 23:1552.
38. Vespa, G. N., L. A. Lewis, K. R. Kozak, M. Moran, J. T. Nguyen, L. G. Baum, and M. C. Miceli. 1999. Galectin-1 specifically modulates TCR signals to enhance TCR apoptosis but inhibit IL-2 production and proliferation. J.Immunol. 162:799.
39. Hughes, R. C. 2001. Galectins as modulators of cell adhesion. Biochimie 83:667.
40. Rabinovich, G. A., A. Ariel, R. Hershkoviz, J. Hirabayashi, K. I. Kasai, and O. Lider. 1999. Specific inhibition of T-cell adhesion to extracellular matrix and proinflammatory cytokine secretion by human recombinant galectin-1. Immunology 97:100.
41. Cooper, D. N. 1997. Galectin-1: secretion and modulation of cell interactions with laminin. Trends Glycosci. Glycotechnol. 9:57.
42. Fiocchi, C. 1998. Inflammatory bowel disease: etiology and pathogenesis. Gastroenterology 115:182.
43. Santucci, L., S. Fiorucci, F. Cammilleri, G. Servillo, B. Federici, and A. Morelli. 2000. Galectin-1 exerts immunomodulatory and protective effects on concanavalin A-induced hepatitis in mice. Hepatology 31:399.
44. Yang, R. Y. and F. T. Liu. 2003. Galectins in cell growth and apoptosis. Cell Mol.Life Sci. 60:267.
45. André, S., S. Kojima, N. Yamazaki, C. Fink, H. Kaltner, K. Kayser, and H. -J. Gabius. 1999. Galectins-1 and -3 and their ligands in tumor biology. Non-uniform properties in cell-surface presentation and modulation of adhesion to matrix glycoproteins for various tumor cell lines, in biodistribution of free and liposome-bound galectins and in their expression by breast and colorectal carcinomas with/without metastatic propensity. J. Cancer Res.Clin.Oncol. 125:461.
46. Targan, S. R., S. B. Hanauer, S. J. van Deventer, L. Mayer, D. H. Present, T. Braakman, K. L. DeWoody, T. F. Schaible, and P. J. Rutgeerts. 1997. A short-term study of chimeric monoclonal antibody cA2 to tumor necrosis factor alpha for Crohn's disease. Crohn's Disease cA2 Study Group. N.Engl.J.Med. 337:1029.
47. Elliott, M. J., R. N. Maini, M. Feldmann, J. R. Kalden, C. Antoni, J. S. Smolen, B. Leeb, F. C. Breedveld, J. D. Macfarlane, H. Bijl, and. 1994. Randomised double-blind comparison of chimeric monoclonal antibody to tumour necrosis factor alpha (cA2) versus placebo in rheumatoid arthritis. Lancet 344:1105.
48. Perillo, N. L., C. H. Uittenbogaart, J. T. Nguyen, and L. G. Baum. 1997. Galectin-1, an endogenous lectin produced by thymic epithelial cells, induces apoptosis of human thymocytes. J.Exp.Med. 185:1851.
49. Rappl, G., H. Abken, J. M. Muche, W. Sterry, W. Tilgen, S. André, H. Kaltner, S. Ugurel, H. -J. Gabius, and U. Reinhold. 2002. CD4+CD7-leukemic T cells from patients with Sezary syndrome are protected from galectin-1-triggered T cell death. Leukemia 16:840.
50. Tsuchiyama, Y., J. Wada, H. Zhang, Y. Morita, K. Hiragushi, K. Hida, K. Shikata, M. Yamamura, Y. S. Kanwar, and H. Makino. 2000. Efficacy of galectins in the amelioration of nephrotoxic serum nephritis in Wistar Kyoto rats. Kidney Int. 58:1941.
51. Lenardo, M. J. 1997. The molecular regulation of lymphocyte apoptosis. Semin.Immunol. 9:1.
52. Adams, J. M. and S. Cory. 1998. The Bcl-2 protein family: arbiters of cell survival. Science 281:1322.
53. Ina, K., J. Itoh, K. Fukushima, K. Kusugami, T. Yamaguchi, K. Kyokane, A. Imada, D. G. Binion, A. Musso, G. A. West, G. M. Dobrea, T. S. McCormick, E. G. Lapetina, A. D. Levine, C. A. Ottaway, and C. Fiocchi. 1999. Resistance of Crohn's disease T cells to multiple apoptotic signals is associated with a Bcl-2/Bax mucosal imbalance. J.Immunol. 163:1081.
54. Wesselborg, S., O. Janssens, and D. Kabelitz. 1993. Induction of activation-driven death (apoptosis) in activated but not resting peripheral blood T cells. J.Immunol. 150:4338.
55. Inohara, H., S. Akahani, and A. Raz. 1998. Galectin-3 stimulates cell proliferation. Exp. Cell Res. 245:294.
56. Sramkoski, R. M., S. W. Wormsley, W. E. Bolton, D. C. Crumpler, and J. W. Jacobberger. 1999. Simultaneous detection of cyclin B1, p105, and DNA content provides complete cell cycle phase fraction analysis of cells that endoreduplicate. Cytometry 35:274.
57. Springer, T. A. 1990. Adhesion receptors of the immune system. Nature 346:425.
58. Springer, T. A. 1994. Traffic signals for lymphocyte recirculation and leukocyte emigration: the multistep paradigm. Cell 76:301.
59. Schwartz, M. A., M. D. Schaller, and M. H. Ginsberg. 1995. Integrins: emerging paradigms of signal transduction. Annu.Rev.Cell Dev.Biol. 11:549-99.:549.
60. Dailey, M. O. 1998. Expression of T lymphocyte adhesion molecules: regulation during antigen-induced T cell activation and differentiation. Crit Rev. Immunol. 18:153.
61. Zell, T., W. J. Kivens, S. A. Kellermann, and Y. Shimizu. 1999. Regulation of integrin function by T cell activation: points of convergence and divergence. Immunol.Res. 20:127.
62. Brewer, C. F. 2002. Binding and cross-linking properties of galectins. Biochim.Biophys.Acta 1572:255.
63. Kuwabara, I. and F. T. Liu. 1996. Galectin-3 promotes adhesion of human neutrophils to laminin. J.Immunol. 156:3939.
64. Ochieng, J., M. L. Leite-Browning, and P. Warfield. 1998. Regulation of cellular adhesion to extracellular matrix proteins by galectin-3. Biochem.Biophys.Res.Commun. 246:788.
65. Michael A. Gitt, Stepehen M. Massa, Hakon Leffler, and Samuel H. Barondes. 1992. Isolation and Expression of a Gene Encoding L-14-II, a New Human Soluble Lactose-binding Lectin. The Journal of Biological Chemistry 267, No. 15. 10601-10606.

1. A method of treating or preventing a disease with impaired apoptosis of T-cells, macrophages and/or antigen-presenting cells or of treating or preventing organ rejection in a patient having undergone organ transplantation, in particular solid organ transplantation, comprising administering galectin-2 or a nucleic acid coding for galectin-2 or its complementary strand, or a nucleic acid hybridizing to such coding nucleic acid or its complementary strand, to a patient in need thereof.

2. The method according to claim 1, wherein said impaired apoptosis, in particular said impaired apoptosis of T-cells is involved in or associated with the pathogenesis of said disease.

3. The method according to claim 1, wherein said disease with impaired apoptosis of T-cells is selected from the group comprising autoimmune diseases and malignant T-cell diseases.

4. The method according to claim 3, wherein said autoimmune diseases are selected from the group comprising rheumatoid arthritis, inflammatory bowel diseases, multiple sclerosis, psoriasis, lupus erythematodes, scleroderma, autoimmune hepatitis and autoimmune nephritis.

5. The method according to claim 3, wherein said malignant T-cell diseases are selected from the group comprising peripheral and lymphoblastic/nodal and extranodal T-non-Hodgkin-lymphomas

6. The method according to claim 4, wherein said inflammatory bowel diseases are Crohn's disease or colitis ulcerosa or indeterminate colitis.

7. The method according to claim 4, wherein said autoimmune disease is rheumatoid arthritis.

8. The method according to claim 1, wherein said galectin-2 is human or rat galectin-2.

9. The method according to claim 1, wherein said galectin-2 has an amino acid sequence selected from the group comprising SEQ ID NO:1 and SEQ ID NO: 2.

10. The method according to claim 1, wherein said galectin-2 is administered in combination with an agent suppressing T-cell proliferation and/or an agent inducing T-cell apoptosis.

11. The method according to claim 10, wherein said agent suppressing T-cell proliferation is selected from the group comprising steroids, macrolides, such as cyclosporin and rapamycin, tacrolimus, azathioprine, 6-mercaptopurine, methotrexate and cyclophosphamide.

12. The method according to claim 10, wherein said T-cell apoptosis inducing agent is selected from the group comprising anti-TNFα-antibody (infliximab, adalimumab and CDP 870), etanercept, leflunamide, natalizumab (anti-Integrin α4β7 mAb), visilizumab (anti-CD3 mAb).

13. The method according to claim 1, wherein said galectin-2 is administered in combination with a drug that induces T-cell apoptosis via a caspase-8 dependent pathway, or wherein said galectin-2 is administered in a patient failing or having failed to show a measurable response to a drug which is known to normally induce T-cell apoptosis via a caspase-8 dependent pathway.

14. The method according to claim 1, wherein said galectin-2 is administered in combination with anti-inflammatory drugs such as 5-Aminosalicylates (5-ASA), corticosteroids, mesalazine, olsalazin, balsalazin, sulfapyridin and non-steroidal anti-inflammatory agent and/or an antirheumatic agent.

15. The method according to claim 14, wherein said antirheumatic agent is a disease modifying anti rheumatic drug (DMARD).

16. The method according to claim 15, wherein said disease modifying anti-rheumatic drug is selected from the group comprising aspirin, naproxen, diclofenac, ibuprofen, naprosyn, indomethacin, piroxican and biological drugs selected from the group comprising anakinra and etodolac.

17. The method according to claim 14, wherein said antirheumatic agent is selected from the group comprising gold compounds, D-penicillamin, antimalaria drugs such as chloroquin, and sulfasalazine.

18. The method according to claim 1, wherein said galectin-2 is administered in combination with cyclo-oxygenase-2-inhibitors (COX-2-inhibitors).

19. The method according to claim 18, wherein said cyclooxygenase-2-inhibitors are selected from the group comprising celecoxib, rofecoxib, and valdecoxib.

20. The method according to claim 1, wherein said galectin-2 is administered in combination with a T-cell activating agent.

21. The method according to claim 1, wherein said galectin-2 is administered in combination with a β-galactoside.

22. The method according to claim 21, wherein said β-galactoside is lactose.

23. The method according to claim 1, wherein said galectin-2 is administered by systemical administration and/or topical administration.

24. The method according to claim 1, wherein said galectin-2 is administered twice daily in an amount of 0.75 mg to 1.5 mg/kg body weight per dose, preferably in an amount of about 1 mg/kg body weight per dose.

25. The method according to claim 1, wherein said administration occurs by ingestion, preferably orally or anally, and/or by injection, preferably by intravenous, intramuscular, intraperitoneal or subcutaneous injection, and/or by nasal application.

26. The method according to claim 1, wherein said galectin-2 is administered as enema and/or as suppository and/or as delayed release dosage form, e.g. encapsulated in a pH dependent release matrix.

27. The method according to claim 1, wherein said galectin-2 is administered in a pegylated or non-pegylated form or as a mixture of the two forms.

28. The method according to claim 1, wherein said patient is one having a pathological condition in which, before administration of galectin-2, a subset of the patient's T-cells and/or a subset of the patient's macrophages and/or a subset of the patient's antigen-presenting-cells fail to undergo apoptosis, preferably adequate apoptosis or wherein a subset of the patient's T-cells and/or macrophages and/or antigen-presenting cells show an impaired or defective apoptosis.

29. The method according to claim 28, wherein said subset of T-cells are T-cells that have previously been activated, preferably via the CD3-pathway or the CD2-pathway or via mitogens, co-stimulatory molecules such as CD28 or CD40, or other pathways such as Toll-like receptors or integrins.

30. The method according to claim 28, wherein said subset of T-cells and/or macrophages and/or antigen-presenting cells are not resting.

31. The method according to claim 30, wherein said subset of T-cells and/or macrophages and/or antigen-presenting cells are not cells that have exited from the cell cycle or are not cells that are arrested in any phase of the cell-cycle.

32. The method according to claim 28, wherein said subset of T-cells and/or macrophages and/or antigen-presenting cells is primarily located in said patient's joints, preferably synovial joints, and/or in said patient's gastrointestinal tract, preferably the lining of said gastrointestinal tract, and/or in said patient's skin, and/or lung and/or liver and/or kidney and/or are a population of peripheral blood cells which are recruited in a mucosa during inflammation.

33. The method according to claim 1, wherein said patient is one having a pathological condition in which, before administration of galectin-2, the ratio between Bcl-2-protein and Bax-protein in T-cells is disbalanced in favour of the anti-apoptotic Bcl-2.

34. A method of treating a patient with an immunodilating agent, comprising administering to a patient in need thereof galectin-2 or a nucleic acid coding for galectin-2 or its complementary strand, or a nucleic acid hybridizing to such coding nucleic acid or to its complementary strand.

35. The method according to claim 34, wherein said galectin-2 or of a nucleic acid coding for galectin-2 or its complementary strand, or a nucleic acid hybridizing to such coding nucleic acid or to its complementary strand, acts as an immunomodulating agent on T-cells and/or macrophages and/or antigen-presenting cells.

36. The method according to claim 35, wherein said T-cells and/or macrophages and/or antigen-presenting cells are human.
